(19) United States
(12) Patent Application Publication
(10) Pub. No.: US 2011/0172621 A1
(43) Pub. Date: Jul. 14, 2011

(54) CLEANABLE AND REUSABLE BODY FLUID-ABSORBING FABRIC PAD

(75) Inventors: Young Kyu Lee, Seoul (KR); Kyung Ju Lee, Seoul (KR); Chang Hwan Lee, Seoul (KR); Eun Hee Kwon, Seoul (KR); Robert Farmer, Toronto (CA); Murray Fischer, Toronto (CA)

(73) Assignee: Welshire Innovative Limited, Dublin (IE)

(21) Appl. No.: 12/684,519

(22) Filed: Jan. 8, 2010

(51) Int. Cl. A61F 13/15

(52) U.S. Cl. 604/365; 604/367; 604/383; 604/385.14; 604/385.15; 604/385.03

(57) ABSTRACT

Disclosed is a cleanable and reusable body fluid-absorbing fabric pad to move moisture including sweat and urine away from the skin of a wearer, the body fluid-absorbing fabric pad including, a diffusive fabric layer coming into contact with the skin of the wearer to remove moisture from the skin, an absorbent non-woven layer coming into contact with the diffusive fabric layer, the absorbent non-woven layer comprising at least 5 wt % of a superabsorbent fiber to remove moisture transported from the diffusive fabric layer, and a waterproof film coming into contact with the absorbent non-woven layer and exhibiting waterproofing properties and breathability. The absorbent non-woven layer is formed by needle-punching or thermally bonding superabsorbent fibers, polyethylene/polypropylene bi-component fibers and rayon fibers, wherein the diffusive fabric layer, the absorbent non-woven layer and the waterproof film are stacked and sewed to form the body fluid-absorbing fabric pad. The body fluid-absorbing fabric pad can utilize constituent fabrics having a multiple sublayer structure of the absorbent layer to increase diffusion rate of moisture from the skin of the wearer.

The present invention relates to an absorbent fabric pad for absorbing body fluids.

### BACKGROUND

Urinary incontinence for individuals includes any involuntary leakage of urine in an unsuitable place at an unsuitable time, as one form of body fluid excretion. This incontinence phenomenon, in particular where involuntary passage of urine results in wetting of undergarments and potentially adjacent clothing and other objects (e.g. chair fabric), may cause loss of confidence, demotivation and social phobia for the affected individuals. Further issues for the individual may include emotional problems such as embarrassment and a sense of shame, a decrease in preferred daily activities, abnormal social relationships and isolation from social activities due to anxiety about urinary incontinence. In addition, undergarments continuously stained with urine may cause skin irritations thereabout, and unpleasant odours resulting from urinary incontinence can be a cause of isolation of the affected individuals from their social circle.

It is also recognised that perspiration is another form of body fluid excretion. For example, in certain athletic sports such as hockey, football, soccer, etc., individuals can become uncomfortable while wearing their athletic gear, in view of their exuding undesirable amounts of perspiration during the course of strenuous athletic activities. Further, repeated exposure of the athletic gear to perspiration requires thorough cleaning of the athletic gear to help kill and take care of any undesirable bacterial growth in the athletic gear and the potential for offensive odours. It is recognised that thorough cleaning of the athletic gear on a regular basis can facilitate the control of the bacteria and odour, however the required frequency of cleaning can result in premature wear of the athletic gear and can be difficult and/or expensive to do on a regular basis.

Other areas where perspiration is an issue, is for those individuals who have abnormally high levels of perspiration outside of strenuous physical activity. An example of this phenomena is where people find themselves in perceived stressful situations, such as in meetings or other social gatherings, and/or their personal metabolism is configured for profuse perspiration even in non-stressful and/or non-strenuous physical situations. In these circumstances, the affected individuals can become embarrassed due to the visible wetness on their clothing (e.g. underarm regions of shirts). Further, repeated excessive perspiration can result in undesirable permanent staining of clothing (e.g. sweat stains), which results in premature disposal of the clothing and the need to purchase replacement clothing.

Accordingly, in view of the above, it is recognised that the undesirable/excessive excretion of body fluids can be encountered by individuals in many forms and situations, and can affect individuals from the very young to the very old. Needed is a reusable absorbent pad (e.g. washable) for use by individuals to help mitigate any of the issues/problems associated and/or otherwise perceived through undesirable/excessive excretion of body fluids. The reusable characteristic of the absorbent pad is desirable to be sufficiently durable to provide for at least a limited number of reuses of the absorbent pad after corresponding repeated washing/cleaning thereof.

In the case of those suffering from urinary incontinence, these individuals can use undergarments with an inserted pad which includes a pad-accepting portion having a predetermined area in a region of the undergarments coming into contact with the pubic region, wherein an absorbent pad is inserted into the pad-accepting portion at a pad-change time, or undergarments with an attached pad, to which a Velcro™ tape-adhered pad is attached, if necessary, for the purpose of treating symptoms without affecting daily life. However, these pad-insertable and pad-attachable undergarments for urinary incontinence have disadvantages in that pads contaminated due to urinary incontinence should be replaced with new ones, and are therefore not compatible with cleaning as they are vulnerable to damage after cleaning and therefore no longer of any use.

A further problem with current state of the art absorbent pads is that they are not designed to be reusable after washing, since the current state of the art disposable absorbent pads use non-cleanable/reusable absorbent polymers (i.e. SAP). For example, it is well known that super absorbent polymers can be super absorbent particles, gels, etc. that can provide a water absorbent, water insoluble, polymeric material in the form of particles/gel. These particle/gel shaped polymers can be made by polymerizing water soluble monomer or monomer blend, e.g., acrylic acid, in the presence of a polyethylenically unsaturated monomer (e.g., N,N′methylenebisacrylamide), copolymerized onto the polymeric backbone to cause crosslinking and to render the polymer insoluble in water. Crosslinking occurs substantially simultaneously with the polymerization. The absorbent polymer is made in its desired final form as beads by reverse phase polymerization, in bulk gel form (e.g. amorphous), or in bulk form and then comminuted to particles. Accordingly, it is recognised that these non-cleanable/reusable absorbent polymers are not usable in applications where cleanable and reusable pads may be desired, as the non-cleanable/reusable absorbent polymers may not reliably/satisfactorily retain their absorbent properties once cleaned.

A further problem with current state of the art absorbent undergarments pads is their inability to suitably retain urine once absorbed, that is, the function to prevent a phenomenon referred to as “rewetting” wherein initially absorbed urine is subsequently released from the absorbent pad, for example in the event of undue pressure being applied to the pad. This function may greatly affect wear comfort or sensations of wearers.

Korean Patent No. 477573 discloses an undergarment for absorbing secretion comprising a body-contact layer wherein an inner pad sewed in general undergarments and integrally formed therewith transports secretion from the body of a wearer to the outside, an absorption-promoting layer closely contacting an outside of the body-contact layer to induce the secretion to move to the outside, and a laminated film layer which is thermally laminated in an outside of the absorption-promoting layer to prevent leakage of the secretion to the outside, wherein the body-contact layer comprises a plurality of pores to allow air to pass there-through and the absorbent fiber layer contains about 20% of Bell Oasis. A disadvantage with this garment is that it does not address the rewetting issue.

U.S. Pat. No. 5,344,698 (entitled “Composite undergarment fabric”) relates to a composite undergarment fabric to moving moisture such as urine away from the skin of the wearer, wherein the composite undergarment fabric comprises a first layer which is made of a hydrophilic material such as polyester and serves to quickly transport urine or body fluids, and a second layer comprising at least 5 wt % of a superabsorbent fiber and a breathable barrier layer applied to the second fabric layer, wherein the first fabric layer and the second fabric layer are formed by knitting a plaited construction. A disadvantage with this garment is that it does not address the rewetting issue.

U.S. Pat. No. 6,658,670 discloses a protective garment comprising a composite structure having a moisture absorber, a moisture barrier covering the moisture absorber, and an outer shell covering the moisture barrier, wherein the moisture barrier has a fibrous matrix incorporating a superabsorbent polymer, wherein the fibrous matrix is disposed between an inner cover and an outer cover, wherein the inner and outer covers is quilted so as to segregate discrete regions of the fibrous matrix. A disadvantage with this garment is that it does not address the rewetting issue.

### SUMMARY OF THE INVENTION

It is an object of the present invention to provide an appropriately configured absorbent pad to mitigate at least some of the above-presented disadvantages.

It is one object of the present invention to provide a body fluid-absorbing fabric pad for repeatedly absorbing moisture and diffusing the same.

It is another object of the present invention to provide a body fluid-absorbing fabric pad for inhibiting rewetting, the phenomenon wherein absorbed urine is leaked out.

Disadvantages of current fabric pads are that they are not suited for repeated washing and reuse and/or they are susceptible to the phenomena of rewetting. Contrary to current fabric pads there is provided a cleanable and reusable body fluid-absorbing pad to move moisture including sweat and urine away from the skin of a wearer, the body fluid-absorbing pad can include; a diffusive fabric layer coming into contact with the skin of the wearer to remove moisture from the skin, an absorbent non-woven layer coming into contact with the diffusive fabric layer, the absorbent non-woven layer comprising at least 5 wt % of a superabsorbent fiber to remove moisture transported from the diffusive fabric layer, and a waterproof film coming into contact with the absorbent non-woven layer and exhibiting waterproofing properties and breathability. The absorbent non-woven layer is formed by needle-punching and/or thermally bonding superabsorbent fibers, polyethylene/polypropylene bi-component fibers, for example, and rayon fibers, wherein the diffusive fabric layer, the absorbent non-woven layer and the waterproof film are stacked and sewed to form the body fluid-absorbing fabric pad. The body fluid-absorbing fabric pad can utilize constituent fabrics having a multiple sublayer structure of the absorbent layer to increase diffusion rate of moisture from the skin of the wearer.

A first aspect provided is a cleanable and reusable body fluid-absorbing pad to move body fluid away from the skin of a wearer, the pad including: an absorbent non-woven layer comprising in total at least 5 wt % of synthetic superabsorbent fibers configured to absorb the body fluid when present adjacent to a wearer toward facing surface of the non-woven layer, the absorbent non-woven layer formed by at least one of needle-punching or thermally bonding of a fiber combination of at least two fiber types selected from the superabsorbent fibers, synthetic multi-component fibers, and rayon fibers.

A further aspect provided is a pad further comprising a water resistant film adjacent to a wearer away facing surface of the absorbent non-woven layer and exhibiting resistance to the transfer of the body fluid through the film, wherein the absorbent non-woven layer and the film are stacked and coupled to form the pad.

A further aspect provided is a pad further comprising a diffusive fabric layer configured for diffusing the body fluid through the diffusive fabric layer and away from contact with the skin of the wearer, the diffusive fabric layer situated adjacent to the wearer toward facing surface of the absorbent non-woven layer, wherein the absorbent non-woven layer and the diffusive fabric layer and the film are stacked and coupled to form the pad.

A further aspect provided is a pad, wherein the absorbent non-woven layer is comprised of a plurality of non-woven sublayers positioned adjacent to one another that are configured to inhibit rewetting of a first sublayer closer to the wearer with body fluid released from a second adjacent sublayer further from the wearer.

A further aspect provided is a body fluid-absorbing fabric pad to move moisture including sweat and urine away from the skin of a wearer, the body fluid-absorbing fabric pad comprising: a diffusive fabric layer coming into contact with the skin of the wearer to remove moisture from the skin; an absorbent non-woven layer coming into contact with the diffusive fabric layer, the absorbent non-woven layer comprising at least 5 wt % of a superabsorbent fiber to remove moisture transported from the diffusive fabric layer; and a waterproof film coming into contact with the absorbent non-woven layer and exhibiting waterproofing properties and breathability, wherein the diffusive fabric layer is formed by weight loss finishing process a double weave structure having one surface woven with a polyethyleneterephthalate (PET) filament and the other surface woven with a divided PET/nylon-conjugated fiber, wherein the absorbent non-woven layer is formed by needle-punching or thermally bonding a superabsorbent fiber, a polyethylene/polypropylene bicomponent fiber and a rayon fiber, and wherein the diffusive fabric layer, the absorbent non-woven layer and the waterproof film are stacked and sewed to form the body fluid-absorbing fabric pad.

A further aspect provided is a cleanable and reusable body fluid-absorbing pad to move body fluid away from the skin of a wearer, the pad including: an absorbent fibrous layer comprising in total at least 5 wt % of synthetic superabsorbent fibers configured to absorb the body fluid when present adjacent to a wearer toward facing surface of the fibrous layer, the absorbent fibrous layer formed by at least one of needle-punching, weaving, or thermally bonding of a fiber combination of at least two fiber types selected from the superabsorbent fibers, synthetic multi-component fibers, and rayon fibers.

A further aspect provided is a body fluid-absorbing fabric pad, wherein the absorbent non-woven layer is a monolayer containing 5 to 50% by weight of the superabsorbent fiber, 50 to 70% by weight of the polyethylene/polypropylene bicomponent fiber and 20 to 45% by weight of the rayon fiber.

A further aspect provided is a body fluid-absorbing fabric pad, wherein the absorbent non-woven layer is a combination of multiple sublayers, wherein each sublayer contains 50 to 100% by weight of the polyethylene/polypropylene bicomponent fiber, 0 to 50% by weight of the superabsorbent fiber, and 0 to 50% by weight of the rayon fiber.

### BRIEF DESCRIPTION OF THE DRAWINGS

The above and other objects, features and other advantages of the present invention will be more clearly understood from the following detailed description taken in conjunction with the accompanying example drawings, in which:

FIG. 1 is a perspective view illustrating a panty for urinary incontinence to as an example article that a body fluid absorbing fabric pad is coupled;

FIG. 2a is a side view of an example coupling of the fabric pad of FIG. 1 to a generic article of wear;

FIG. 2b is a further embodiment of the example coupling of FIG. 2a;

FIG. 3a is a side view of an example coupling between adjacent layers of the fabric pad of FIGS. 2a and 2b;

FIG. 3b is a top view of the example coupling between adjacent layers of the fabric pad FIGS. 3a;

FIG. 4 shows an example configuration of multiple fiber types in the absorbent layer of the pad of FIG. 1;

FIG. 5 shows an example multiple sublayer structure of the absorbent layer of FIG. 4;

FIG. 6 shows a two sublayer embodiment of the absorbent layer of FIG. 5; and

FIG. 7 shows a three sublayer embodiment of the absorbent layer of FIG. 5.

### DETAILED DESCRIPTION OF THE EMBODIMENTS

### Applied Uses of Absorbent Pad 12

### SAF Verses SAP

### SAP

A problem with current state of the art absorbent pads is that they are not designed to be reusable after washing, since the current state of the art disposable absorbent pads use non-fiber absorbent polymers (SAP). For example, it is well known that super absorbent polymers can be super absorbent non-fibers (e.g. particles, gels, etc.) that can provide a water absorbent, water insoluble, polymeric material in the form of particles. These non-fiber polymers can be made by polymerizing water soluble monomer or monomer blend, e.g., acrylic acid, in the presence of a polyethylenically unsaturated monomer (e.g., N,N′methylenebisacrylamide), copolymerized onto the polymeric backbone to cause crosslinking and to render the polymer insoluble in water. Crosslinking occurs substantially simultaneously with the polymerization. The absorbent polymer is made in its desired final form as beads by reverse phase polymerization, in bulk gel form (e.g. amorphous), or in bulk form and then comminuted to particles. Accordingly, it is recognised that these non-fiber absorbent polymers (i.e. SAP) are not usable in applications where cleanable and reusable pads may be desired, as the non-fiber absorbent polymers (SAP) may not reliably retain their absorbent properties once cleaned. It is recognised that the physical shape/dimensional characteristics of SAP are such that the SAP does not have a predominant length (as compared to its width) as compared to the physical shape/dimensional characteristics of SAF that does have a predominant length (as compared to its non-dominant width).

Further, particulate material in SAP, e.g. powder, granular absorbent polymer) can have a greater tendency to form clumps when they have absorbed body fluid, as compared to fibrous material in SAF that may not tend to form clumps when wettened (e.g. have absorbed body fluid). Further, super absorbent gels can be defined as a solid, jelly-like material that can have properties ranging from soft and weak to hard and tough.

Further to the above, it is recognised that the absorbent polymer content of SAP (e.g. particles/gells) does not contribute to the woven, matted (non-woven), and/or twisted fibrous nature of the layer 2.

### SAF

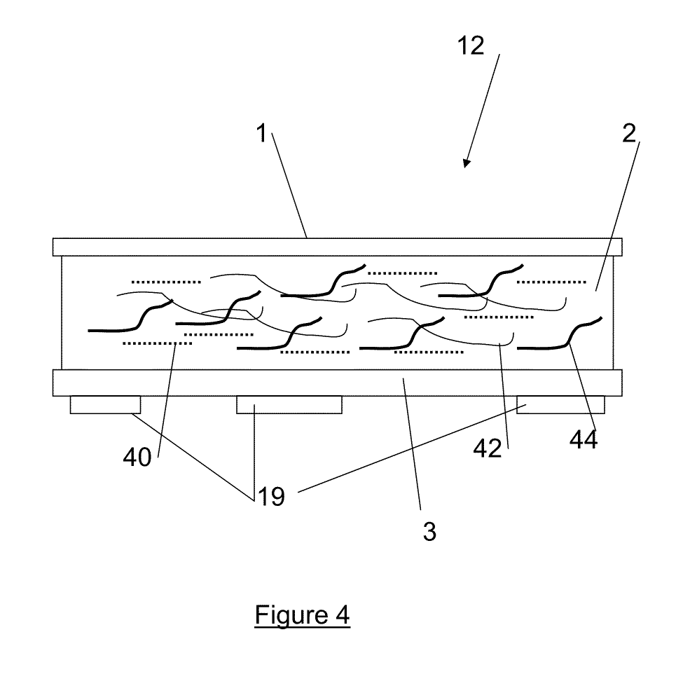

It is recognised that super absorbent fiber (SAF) polymers (e.g. an example of a component 40 of an absorbent layer 2 of a pad 12—see FIG. 4) are substantially different from non-fiber absorbent polymers (also known as SAP), as super absorbent fibers provide for a fluid-absorbent fluid-insoluble fiber/filament. In other words, SAF can be defined as a body fluid insoluble but body fluid swellable (upon body fluid absorption) fibrous super absorbent polymer. It is recognised that the swelling of the SAF will decrease upon cleaning of the layer 2 to remove a substantial portion of the absorbed body fluid from the layer 2 to a recognised reduction level perceived as “clean” by the wearer 8. the body fluid can be defined as an aqueous solution.

The SAF material is considered by the Applicant as a more stable material (over that of SAP) and therefore is more able to retain its fiber/filament structure on absorbing water, that is to say the SAF material retains its fibrous nature upon fluid absorption. Applicant has found that the use of SAF over SAP in the layer 2 contributes to the clean ability and reusability of the layer 2. Applicant has found that SAF is a body fluid insoluble but body fluid swellable fibrous polymer that can be made reliably by large scale manufacturing processes and that the SAF have satisfactory absorbency properties compared to the absorbency properties of conventional particulate/gell water swellable polymers SAP.

One example of SAF is staple fiber produced by extruding a solution of a polymer through a spinneret to form continuous filaments, gathering the continuous filaments to form a tow and continuously cutting the filaments in a cutter to form the staple fiber 40 for use as a component 40 of the layer 2 in a selected percent weight value. Accordingly, a super absorbent fiber (SAF) can be defined as a water absorbent water insoluble fiber and/or filament having a selected length or lengths, having a selected gel capacity to provide for desired fluid absorption properties. In one aspect, a water absorbent, water insoluble polymeric element is an elongated shaped (e.g. fiber, filament) such that the length of the shape is; at least 2 times as long as its width, at least 3 times as long as its width, at least 4 times as long as its width, at least 5 times as long as its width, at least 6 times as long as its width, at least 7 times as long as its width, at least 8 times as long as its width, or at least 9 times as long as its width, for example.

An example process for making the SAF is where substantially immediately after extruding or otherwise shaping the polymer solution, the linear polymer reagent forms a uniform solid mixture in the form of an article of the desired shape. The article is initially very soft because of the presence of residual solvent, e.g., water. By soft is meant that the article can be elongated or its shape can be changed relatively easily when external forces are applied. The conversion of the liquid solution to the soft solid articles is by precipitation and involves solvent evaporation, solvent extraction, solvent sublimation, or insolubilizing the polymer. In the case of SAF spinning process, the SAF made is collected as either individual fiber, such as staple fiber or filament. The term “line” can be used to refer to the shaped article/element formed by forcing the polymer solution through a die orifice. Spunbonded fibers” can refer to small diameter fibers that are formed by extruding molten thermoplastic material as filaments from a plurality of fine capillaries of a spinneret. Such a process is disclosed in, for example, U.S. Pat. No. 4,340,563 to Appel et al. The fibers may also have shapes such as those described, for example, in U.S. Pat. No. 5,277,976 to Hogle et al. which describes fibers with unconventional shapes.

Another optional type of SAF is formed by hydrolysing the outer surfaces of polyacrylonitrile (or other substrate material) fibres so as to form a sheath of fibrous fluid soluble polymer and a core of fluid insoluble polymer that gives the fibre strength.

It is also recognised that concerning SAF versus SAP, the absorbent element of the polymer for SAF is in a thread or resembling a thread-like shape, having an elongated shape with a length that is significantly greater than the width. Fiber can be defined as a class of materials that are continuous filaments or are in discrete elongated pieces, similar to lengths of thread. Further, it is recognised that absorbent polymer content of SAF can be oriented (aligned in direction), woven, matted (e.g. non-woven), twisted, etc, as compared to the absorbent polymer content of SAP (e.g. particles/gells) that cannot be woven, matted (non-woven), twisted, etc. Therefore, the absorbent polymer content of SAF (e.g. fibers/filaments) does contribute to the woven, matted (non-woven), and/or twisted fibrous nature of the layer 2, i.e. is the super absorbent polymer (i.e. swellable fiber/filament) that adds to the cohesion of the layer as a non-woven mat, as a woven mat, or as a combination of woven and non-woven mats.

Further, it is recognised that the SAF can be made out of synthetic polymers or man-made fibers (man-made fibers that come from natural raw materials). Synthetic or man-made SAF may come from natural or synthetic materials such as petrochemicals, wherein it is the fibers themselves that swell upon absorption of body fluid.

### PAD 12

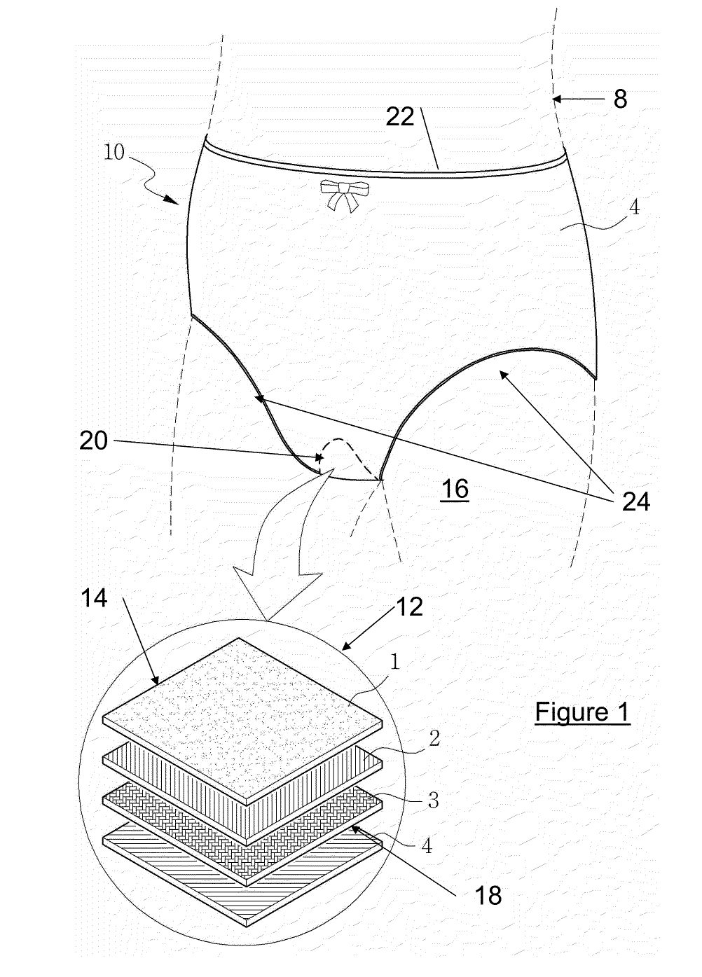

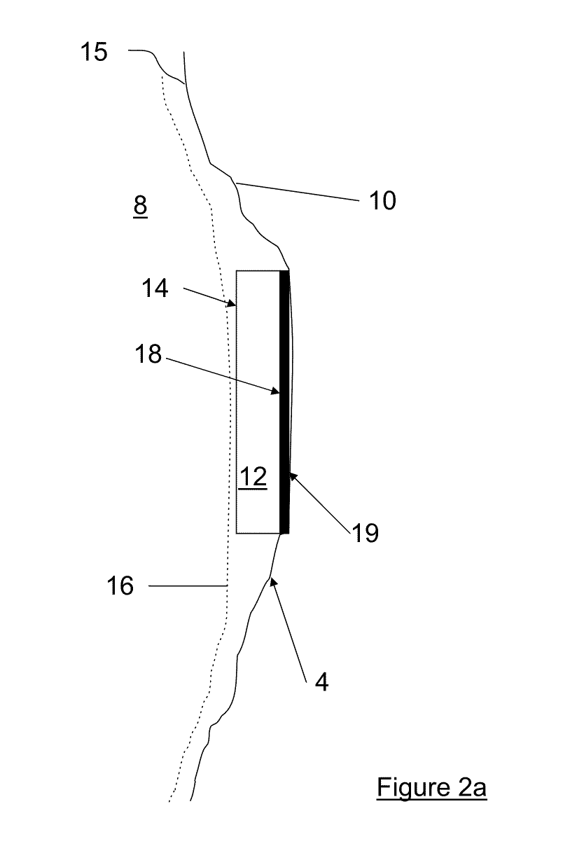

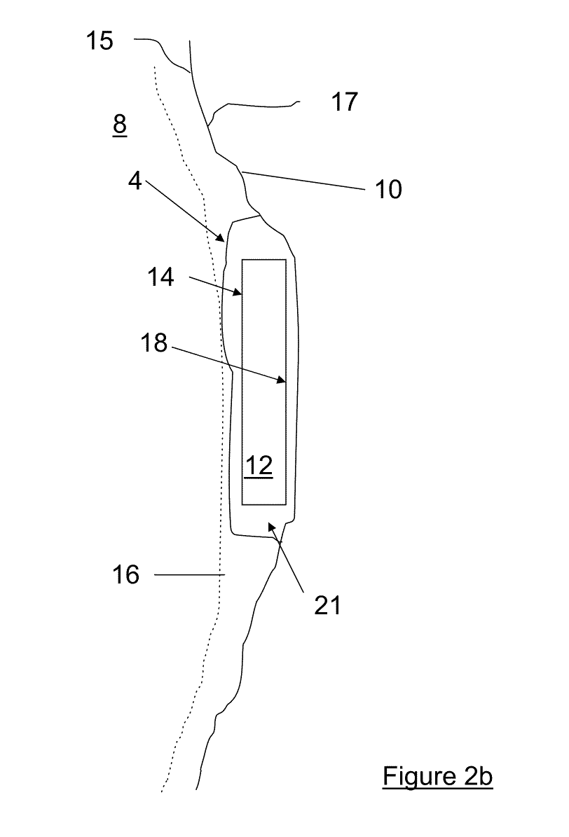

Referring to FIG. 1, shown is an absorbent pad 12 in expanded view having an interior surface 14 (an example of a wearer toward facing surface) for positioning adjacent to skin 16 (for example) of an individual wearer 8 (shown in ghosted view) and an exterior surface 18 (an example of a wearer away facing surface) for positioning adjacent to material 4 (e.g. cloth) of a wearer's clothing/undergarment 10 (e.g. in the crotch region of underwear as shown by example only). The pad 12 is configured for absorbing exuded body fluid from the wearer 8, such as but not limited to perspiration and/or urine. It is recognised that the wearer's article 10 can be considered as part of a category encompassing a wide variety of materials that are used to cover one or more portions of wearer's 8 body such as but not limited to: footwear such as a boot or shoe; athletic equipment including for example protective pads, shirts, pants, etc.; general protective equipment such as gloves, helmets or other head gear, jackets, pants, etc.; an undergarment such as a panty or an undershirt; clothing such as socks, support garments such as Spanx™ and/or pantyhose, shirts, pants, etc.; and other articles suitable for wear by the individual 8. For the sake of simplicity only, the wearer's article 10 will hereafter be referred to generically as an article of wear 10. In any event, it is recognised that the pad 12 is configured for positioning adjacent to an interior surface 15 (as compared to an exterior surface 17) of the article of wear 10, see FIG. 2b.

Referring again to FIG. 1, it is recognised that the absorbent pad 12 is washable/cleanable and reusable, being able to endure a limited number of wash/dry cycles, as further described below, e.g. for example a repeated number of wash and dry cycles (in order to substantively remove the absorbed body fluid and to reenergize the absorbent layer 2 to be reworn by the wearer 8 and for the pad 12 to be further used to accept, absorb, and retain as designed further body fluid produced by the wearer 8 during the reuse (e.g. after cleaning) if the pad 12 and/or core layer 2 (if separable from the other components of the pad 12 for cleaning). Cleaning can take a number of forms including methods such as but not limited to washing with water and detergent and drying, rinsing with water and drying, and known dry cleaning methods. The drying portion of the cleaning can be assisted dry mechanical means and/or air drying. In the case of women's panties 10, as one example use of the absorbent pad 12, typical construction includes two or more fabric 4 pieces (e.g. a front and a rear fabric 4 piece) joined by seams at the crotch and sides. The crotch fabric is sized to cover the genital area and contains the absorbent pad 12 fastened to the crotch fabric 4 in association with a gusset 20. Typical construction also includes a waistband 22 (often elastic) and a pair of leg openings 24 (also often elastic). It is recognised that the pad 12 can be cleanable (e.g. using water and/or other dry cleaning methods) and therefore reusable once cleaned (e.g. previously collected/absorbed body fluids have been removed from the layers 1,2 as a result of the cleaning).

The article of wear 10 can be in a form of underwear, usually light and snug-fitting, designed to be worn by women or girls 8 in the area directly below the waist of the individual 8. It is recognised the gusset 20 can be defined as a triangular, square, rectangular, or other appropriately shaped piece of fabric inserted into a seam of the article of wear 10 to add breadth or reduce stress in the seam area. Gussets 20 can be at the underarms of traditional shirts 10 and chemises 10 to help shape the article of wear 10 to the body of the wearer 8. As well, gussets are used in manufacturing of modern tights 10 or pantyhose 10 to add breadth at the crotch seam.

### Basic Components of Absorbent Pad 12

Referring again to FIG. 1, the absorbent pad 12 can include three primary layers of: an interior layer 1 (having the interior surface 14) for positioning adjacent to the wearer 8 and facilitating the transfer of body fluid away from the skin 16 of the wearer 8 and into the pad 12; an intermediate or median layer 2 for facilitating absorption of the body fluid transferred though the first layer 1; and an exterior layer 3 (having the exterior layer 18) for positioning adjacent to the interior surface 15 Of the article of wear 10 (see FIG. 2a) for inhibiting transfer of the absorbed body fluid into the adjacent material 4 of the article of wear 10.

For example, the absorbent fabric pad 12 comprises a diffusive fabric layer 1 for coming into contact with the skin 16 of the wearer 8 to remove moisture such as urine from the wearer's skin 16, an absorbent non-woven layer 2 coming into contact with the diffusive fabric layer 1 and comprising at least 5 wt % of a superabsorbent fiber to remove moisture transported from the diffusive fabric layer 1, and a waterproof film layer 3 coming into contact with the absorbent non-woven layer 2 and exhibiting waterproofing properties and breathability. These three layers are stacked and sewn in an outer cloth 4 of a panty 10 for urinary incontinence 10, when they are used for the panty 10 for urinary incontinence.

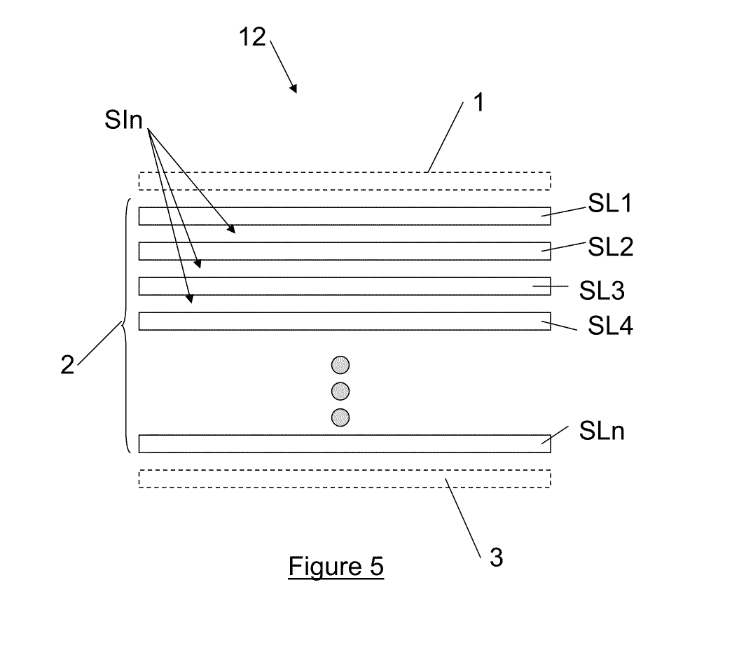

For example, the body fluid-absorbing fabric pad 12 utilizes constituent fabrics having a multiple-layer structure (e.g. each of sublayers of the absorbent layer 2 having different porosities) to increase a diffusion rate of body fluid into a selected sublayer or selected sublayersof the layer 2 of the pad 12, such that the diffusive fabric layer 1 comes into contact with the skin 16 of the wearer 8 to most rapidly (for example in comparison to any of the other layers 2 of the pad 12) transfers body fluid from the wearer 8. In addition, the absorbent layer 2 is composed of a non-woven layer, which is formed by needle-punching or thermally bonding a combination of the superabsorbent fibers 40, the polyethylene/polypropylene bi-component (or multi-component) fibers 42, and the rayon fibers 44 to facilitate maximized absorbability of the body fluid. For example, the absorbent non-woven layer 2 may be a monolayer containing 5 to 50% by weight of the superabsorbent fibers 40, corresponding 50 to 70% by weight of the multi-component fibers 42, and corresponding 20 to 45% by weight of the rayon fibers 44, in terms of the total fibers weight of the layer 2 (e.g. a total of all the fiber type weights in all of the sublayers SLn—see FIG. 5).

### Interior Layer 1

The interior layer 1 can be configured for positioning adjacent to the skin 16 of the wearer 8, is porous, and can have hydrophilic and/or hydrophobic characteristics that inhibit any body fluid waste (discharged/exuded from the wearer 8) from collecting between the wearer's skin 16 and the interior layer 1. In other words, the layer 1 facilitates the collection of fluid adjacent to the surface 14 (e.g. near the skin 16 of the wearer 8) and subsequent dispersion/transfer of the collected fluid through to the surface 34 of the layer 1. Hydrophilic can refer to a property of the layer 1 to strongly attract (e.g. predisposed to fluid attraction) fluid positioned adjacent (e.g. near the surface 14) to the layer 1. Hydrophobic can refer to a property of the layer 1 to strongly repel water away from it's surface (e.g. surface 34) towards the absorbent layer 2 for subsequent absorption. Accordingly, the material (e.g. fabric) comprising the layer 1 has a property of transition/transfer ability in which the fluid is transmitted through the material cross-section of the fabric layer 1. In any event, the interior layer 1 is pervious to the body fluids and is configured for facilitating the collection of the body fluid from adjacent to the wearer 8 and towards the absorbent layer 8, for ultimate absorption by one or more layers SLn.

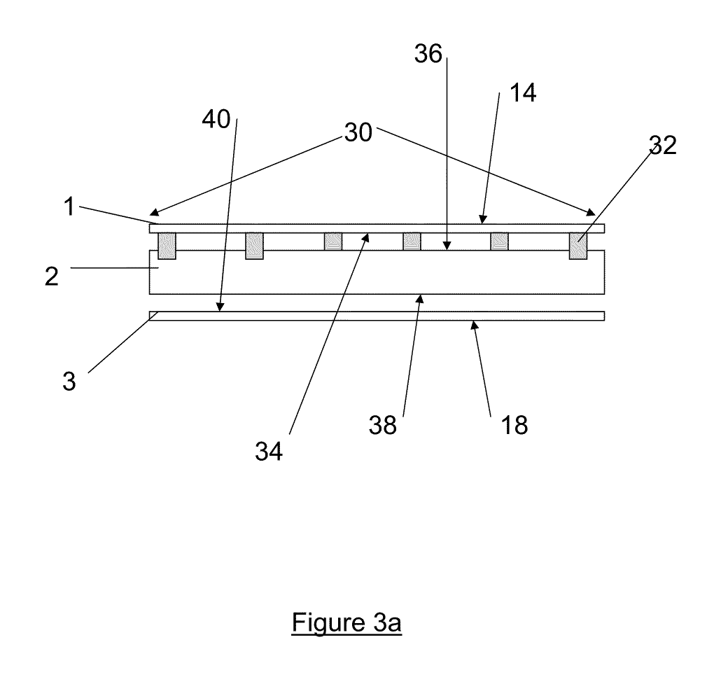

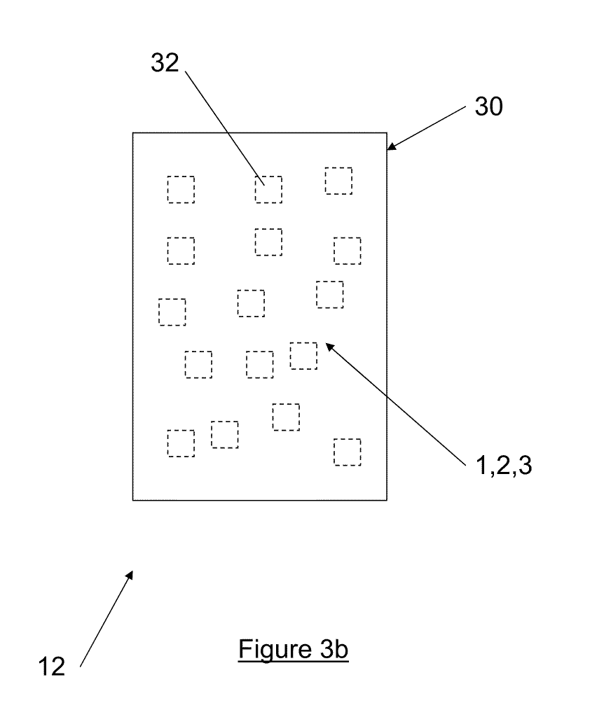

This layer 1 can be used to facilitate protection of the absorbent layer 2 from mechanical damage (e.g. due to washing and/or wear by the wearer 8). This layer 1 firstly faces the produced/excreted body fluid and diffuses the body fluid, in contact with the exterior surface 14 (see FIG. 3a), through the layer 1 and into the layer 2 arranged thereon i.e., the absorbent non-woven layer 2. The absorbance rate and/or diffusion rate properties of the layer 1 can be designed to inhibit collection of the body fluid between the layer 1 and the wearer 8 (e.g. skin 16). One example of the material comprising this layer 1 is a double weave structure having one surface woven with a polyethyleneterephthalate (PET) filament and the other surface woven with a divided PET/nylon-conjugated fiber and being subjected to weight loss finishing. This is a fabric layer 1 exhibiting moisture-permeability, for an example see Korean Patent No. 333125, and is commercially available under the trademark “Aquatrans®”, also described in U.S. Pat. No. 6,381,994.

It is also recognised that the exterior layer 1, if present in the pad 12, can be comprised of other material(s) (other than as described above) that have desired body fluid transference properties for facilitating moving the body fluid away from the wearer 8 and into the absorbent layer 2. The transfer of the body fluid through the material composition of layer 1 can be defined as a transport phenomenon using various mechanisms by which the body fluid moves from the interior surface 14 to the outside/exterior surface 34 of the layer 1. One transport phenomena is conduction through the material of the layer 1, thereby encouraging a flow of the body fluid between the surfaces 14, 34 in the direction towards the layer 2. The transport of the body fluid can be affected by the presence of external sources, such as body fluid pressure (e.g. fluid pressure due to expulsion of the body fluid by the wearer 8), and/or the amount (e.g. build up) of body fluid present adjacent to the surface 14, and/or level of already absorbed body fluid in the layer 2.

### Absorbent Layer 2

The intermediate/median layer 2 is interposed between the interior layer 1 and the exterior layer 3 and can be coupled, as further described below, to the interior layer 1 at discrete locations over the surface at the interface there-between and/or the exterior layer 3 (e.g. using an adhesive film at the interface between the intermediate/median layer 2 and the exterior layer 3). The intermediate/median layer 2 is formed of synthetic fibers having fluid absorption dispersion and retention characteristics, for absorbing, dispersing, and retaining the body fluid passing through the interior layer 1 and down into the intermediate/median layer 2. The intermediate/median layer 2 can also be referred to as an absorbent layer 2. The main physical properties of this layer 2 are absorbance rate, absorbance, and retention amount of the body fluid. It is recognised that the layer 2 can be comprised of a number of fibers 40,42,44 that provide for a mat of woven, non-woven, and/or combined woven and non-woven cohesive fibrous structure, such that the fibers 40 constitute SAF.

The absorbent layer 2 includes non-woven synthetic fibers comprising at least 5 wt % of a superabsorbent fiber 40 to absorb and retain body fluid transported from the layer 1 (or wearer 8 in the case where the layer 1 is absent from the pad 12). The absorbent non-woven layer 2 is formed by needle-punching and/or thermally bonding the superabsorbent fibers 40 with multi-component (e.g. bi-component fibers) 42 (e.g. polyethylene/polypropylene) and rayon fibers 44 (see FIG. 4), provided in the layer 2 as a composite fiber 40,42,44 mixture. For example, the multi-component fibers 42 are dispersed throughout the layer 2 and can be thermally and/or mechanically (e.g. twisted and/or bent/kinked around one another) bonded with one another to promote cohesion of the fibers 40,42,44 layer 2 combination/composition. As well, the rayon fibers 44 can also be dispersed throughout the layer 2 to promote cohesion between the fibers 40,42,44 present in the layer 2.

Super-absorbent fibers 40 have the property of absorbing moisture up to many times its original weight, undergoing measurable expansion during absorption, and eventually becoming a gel. Super-absorbent fibers 40 are a type of fiber that can absorb a large amount of fluid quickly and is different from normal non-super absorbent fibers in that rather than using physical absorption alone, the super absorbent fibers absorb including chemical absorption. The total absorbency and swelling capacity are controlled by the type and degree of cross-linking to the polymer. Low density cross-linked super absorbent polymers (SAP) generally have a higher absorbent capacity and swell to a larger degree.

These types of SAPs also have a softer and more cohesive gel formation. High cross-link density polymers exhibit lower absorbent capacity and swell. The gel strength is firmer and can maintain particle shape even under modest pressure. Other materials used to make a superabsorbent polymer re materials such as but not limited to; polyacrylamide copolymer, ethylene maleic anhydride copolymer, cross-linked carboxy-methyl-cellulose, polyvinyl alcohol copolymers, cross-linked polyethylene oxide, and starch grafted copolymer of polyacrylonitrile.

The term “superabsorbent fiber (SAF)” as used herein can be defined, for example, as a material which is capable of containing 20 times the weight thereof in moisture and exhibits desirable wearing sensations, since it is free of fiber balling generated in conventional absorbers. SAF is available under the trade name “Oasis” from Technical Absorbents Ltd. of England. Super absorbent fibers 40 are manufactured as, for example: discontinuous fibers coated with a binder material with the binder material adhering the fibers to super absorbent particles; fibers are substantially unbonded except to the super absorbent particles; the binder may be present at an amount which is sufficient to substantially continuously coat the fibers; plural coatings of various binder materials may be used; the binder material may be heat fusible or heat curable and the treated fibers mixed with other fibers for use in producing a wide variety of different super absorbent properties. Polyacrylic acids (polyacrylate) is an example polymer employed for super absorbent fibers 40. Super-absorbent synthetic polymers are used in the fibers 40 as an absorbent for water and aqueous solutions (e.g. body fluids), and are therefore different from traditional absorbent natural (i.e. non-synthetic) materials such as cloth, cotton, paper wadding, and cellulose fibers.

The multi-component fiber 42 is comprised of two or more polymers of different chemical and/or physical properties extruded from the same spinneret with each of the (e.g. both in the case of a two component fiber 42) polymers within the same filament/strand/fiber 42. In the layer 2, the multi-component fibers 42 can provide thermal bonding, self bulking, very fine/thin dimensioned fibers, unique cross sectional shapes of each of the polymers in the fiber, and functionality of special polymers or additives at reduced cost. Examples of bi-component configurations for the multi-component fibers 42 are such as but not limited to: a sheath/core; side-by-side; and/or eccentric sheath/core arrangements. It is recognised that similar arrangements can be done with multi-component fibers having more than two polymers.

Advantages of multi-component fibers 42 as thermal binder fibers in the layer 2 are such as but not limited to: uniform/defined distribution of adhesive in the layer 2; the fibers 42 remain/form a part of structure for the fiber 40,42,44 mixture and add mechanical integrity for the fiber 40,42,44 mixture; use of customized sheath polymer materials to bond various fibers 40,42,44 to one another; a wide range of bonding temperatures dependent upon the polymers used in the multi-component fibers 42; provision for use of acceptable recyclable polymers in the layer 2 composition; and provide lamination/moulding/densification of composites. Example polymer combinations in the fibers 42 include combinations such as but not limited to: polyester core (e.g. 250 C melt point) with co polyester sheath (e.g. melt points of 110 C to 220 C); polyester core (e.g. 250 C melt point) with polyethylene sheath (e.g. 130 C melt point); and/or polypropylene core (e.g. 175 C melt point) with polyethylene sheath (e.g.130 C melt point). Other example compositions are such as but not limited to: polyester side by side; polyethylene side by side; polypropylene side by side; polyethylene sheath with polyethylene core; polypropylene sheath with polypropylene core; polypropylene sheath with polyethylene core; polyethylene sheath with polypropylene core; and/or side by side polyethylene and polypropylene. Further, it is recognised that self bulking multi-component fibers 42 can be created with side-by-side or eccentric cross section and variation in orientation across the fiber 42 inhibits crimping due to differential shrinkage or strain with applied heat or relaxation.

The other composite component of the layer 2 is rayon fibers 44. Rayon fibers 44 are manufactured from regenerated cellulose fiber. Because rayon fibers 44 are produced from naturally occurring polymers, rayon fibers 44 are neither truly synthetic fibers nor natural fibers; rayon fibers 44 are semi-synthetic fibers. Rayon fibers 44 facilitate for properties in the layer 2 of softness, smoothness, coolness, comfortableness, and highly absorbent, but they also inhibit the insulation (i.e. retaining or otherwise insulating) of body heat as compared to natural fibers.

### Exterior Layer 3

The exterior layer 3 is positioned adjacent to the intermediate/median layer 2 and is non-porous to the body fluid, thereby inhibiting the transfer of any body fluid resident in the intermediate/median layer 2 from penetrating through the exterior surface 18 of the pad 12 and thereby seeping into the material 4 of the article of wear 10. It is recognised that the exterior layer 3 may be breathable, thus providing for evaporation of the body fluid (e.g. as vapour) from the absorbent layer 2 while inhibiting transfer of the body fluid as fluid through the exterior layer 3.

For example, a waterproof/resistant film 3 can be a waterproofing/resistant breathable film which inhibits body fluid contained in the layer 2 from penetrating to the material 4 of the article of wear 10 and can also allow outside air to pass there-through. This layer 3 can be obtained from companies such as Daemyung Chemical Co., Ltd., Hanjin P&C Co., Ltd., Hans Industrial Co., Ltd, etc.

### Optional Releasably Securable Fastener 19

Referring again to FIG. 4, releasable fasteners 19 can be used to releasably attach the pad 12 to the article of wear 10 (see FIG. 1) between the exterior surface 18 of the pad 12 and material 4 of the interior surface 15 of the article of wear 10. For example, the releasable fastener 19 can be Velcro™ (e.g. fabric hook-and-loop fasteners) for use in releasably securing the pad 12 to the article of wear 10, such that compatible strips of Velcor™ are secured to the surface 18 and the surface 15, thus facilitating releasable connection of the pad 12 in the interior of the article of wear 10. After use of the pad 12, the wearer 8 can remove the pad 12 from the article of wear 10 and wash/clean the pad 12 separately from the article of wear 10, as desired. Also, the releasable fastener 19 can be referred to as a slidingly engaging fastener 19, where instead of loops and hooks, the releasable fastener 19 has interlocking islands with undercut edges that slide together (available at Material ConneXion). Other options for the releasable fastener 19 include means of fastening such as but not limited to snaps, buttons, zippers, etc.

### Alternative Layer Configurations

Further to the above, it is recognised that the pad 12 can be comprised of a fewer number of layers that as described above. For example, the pad 12 can be comprised only of the absorbent layer 2 without having any of the layers 1,3, can be comprised of the absorbent layer 2 and only the diffusive interior layer 1, or can be comprised of the absorbent layer 2 with only the fluid transfer resistant exterior layer 3, as desired. For example, the pad 12 can be configured for placement in an enclosure 21 (see FIG. 2b) of the article of wear 10 and therefore may only have in it's construction the absorbent layer 2, the absorbent layer 2 and only the diffusive interior layer 1, or the absorbent layer 2 with only the fluid transfer resistant exterior layer 3.

### Example Embodiments of Pad 12

The body fluid-absorbing fabric pad 12 is attached to an undergarment 10, such that the undergarment 10 has similar slimness, as a liner attached to the undergarment in the crotch region and is worn in such a way as to be hidden from view. The pad 12 comprises at least one absorbent layer 2. In addition, the absorbent fabric pad 12 is configured to be reusable for a limited number of washes/cleanings, i.e. not a one-time use disposable product, and is therefore reusable for at least a limited number of times (e.g. 50 times) after washing, while experiencing limited deterioration in absorbance properties.

The pad 12 is advantageously suitable for wearing for a long period after and before parturition, exercise, climbing and long-distance driving, thus inhibiting the necessity of changing undergarments 10 or replacing pads 12, and may be utilized in a variety of applications including such as but not limited to; armpits, breastfeeding, as well as for urinary incontinence.

In one embodiment, the body fluid-absorbing pad 12 is configured to move moisture including sweat and/or urine away from the skin 16 of the wearer 8. The diffusive layer 1 is configured as a fabric layer formed by a weight loss finishing process as a double weave structure having one surface woven with a polyethyleneterephthalate (PET) filament and the other surface woven with a divided PET/nylon-conjugated fiber. The absorbent layer 2 in the pad 12 is positioned into contact with the diffusive fabric layer 1 and includes one (or more layers) as an absorbent non-woven layer comprising at least 5 wt % of a superabsorbent fiber to remove moisture transported from the diffusive fabric layer 1. The absorbent non-woven layer 2 is formed by needle-punching and/or thermally bonding superabsorbent fibers 40, polyethylene/polypropylene bi-component fibers 42, and rayon fibers 44 (see FIG. 4. It is recognised that the fibers 40,42,44 can comprise only synthetic fibers that are suitable for repeated exposure to water and detergents for cleaning.

The exterior layer 3 is a waterproof film coming into contact with the absorbent non-woven layer 2 and exhibiting waterproofing properties and breathability, wherein the diffusive fabric layer 1, the absorbent non-woven layer 2 and the waterproof film 3 are stacked and sewed/glued to form the body fluid-absorbing fabric pad 12.

The thickness and weight ranges of respective layers 1,2,3 are as follows, for example: diffusive fabric layer 1 of 0.5-0.9 mm, weight 80-200 g/m2; absorbent non-woven layer 2 of 1.4-1.8 mm, weight 90-200 g/m2; waterproof film 3 of 0.2-0.4 mm, weight 18-22 g/m2. Further, the material 4 of the article of wear 10 can be in the range 0.3-0.8 mm, weight 50-150 g/m2. The afore-mentioned layers 1,2,3, i.e., diffusive fabric layer 1, absorbent non-woven layer 2 and waterproof film 3 are stacked and sewed in a predetermined region of the article of wear 10, for example.

### Example Connection Between Layers 1,2,3

Referring to FIGS. 3a and 3b, In particular, the interior 1 and intermediate/median 2 layers can be coupled to one another at their interface at a plurality of coupling 32 locations positioned over the surface of the interface, e.g. at a matrix/grid (e.g. of regular and/or random spacing) of connection 32 locations, so as to inhibit any interference with the transfer of body fluid from the interior layer 1 seeping down into the intermediate/median layer 2. Further, the coupling between the interior 1 and intermediate/median 2 layers is done so as to minimize the gap distance between an inside surface 34 of the interior layer 1 and an inside surface 36 of the intermediate/median layer 2.

One embodiment of the coupling 32 between the interior 1 and intermediate/median 2 layers is a number of distributed adhesive (e.g. hot melt glue) dots 32 that are used to couple layers 1,2 to one another. The adhesive is applied in the grid pattern having a defined dot 32 size and a defined inter-dot 32 spacing. Preferably, the adhesive does not penetrate from the inside surface 36 through the thickness to an outside surface 38 of the intermediate/median layer 2, rather the adhesive dots 32 remain localized near the inside surface 36 of the intermediate/median layer 2. It is recognised that the adhesive dots 32 can penetrate a certain distance partway into the thickness of the layer 2, for example.

A second embodiment of the coupling between the interior 1 and intermediate/median 2 layers is a number of distributed stitching locations 32 that are used to couple the interior 1 and intermediate/median 2 layers to one another. It is recognised that each of the stitches 32 used to couple the layers 1,2 do not penetrate from the inside surface 36 through the thickness to the outside surface 38 of the intermediate/median layer 2, which would provide a plurality of undesirable short circuit paths for the body fluid through the thickness of the intermediate/median layer 2 to the exterior layer 3. Rather, the stitches 32 are inserted through the interior layer 1 and then partway through the thickness of the intermediate/median layer 2. It is also recognised that the coupling between the interior 1 and intermediate/median 2 layers can be a combination of distributed stitching locations 32 and adhesive dots 32, as desired.

Further, the layers 2,3 can be attached to one another using adhesive and/or stitching between the outside surface 38 of the layer 2 and the inside surface 40 of the layer 3.

Further, any of the layers 1,2,3 in the stack of layers 1,2,3 can be connected to one another about a periphery 30 of the pad 12, such as but not limited to by using stitching through the stacked layers 1,2,3 and/or adhesive between the layers 1,2,3 about the periphery 30. This connection about the periphery 30 can be present in absence and/or in addition to the use of the connections 32 distributed between the layers 1,2. This connection about the periphery 30 can be present in absence and/or in addition to the use of connection (e.g. adhesive film) between the layers 2,3.

Manner of Coupling of Absorbent Pad 12 with Article of Wear 10

The pad 12 can be coupled (e.g. permanently affixed or releasably secured) to the article of wear 10 in a desired location, i.e. placed so as to provide for contact with the wearer's 8 skin 16 (for example) when the article of wear 10 is worn by the wearer 8. It is also recognised that the pad 12 may not contact the skin 16 directly, rather the pad 12 can be placed adjacent to relatively thin fabric 4 or other porous material 4 of the article of wear 10 (see FIG. 2b, e.g. inside of a pocket/enclosure 21 of the article of wear 10, such that the pocket 21 is positioned/adjacent to the wearer 8 such as the skin 16 with a layer of the material 4 positioned between the skin 16 and the pad 12). Therefore, the pad 12 can be located directly adjacent to the skin 16 of the wearer 8 and/or can be located inside of a pocket or other material enclosure 21 of the article of wear 10 (such that there is a porous material 4 situated between the pad 12 and the skin 16).

Referring to FIG. 2a, one example of coupling the pad 12 to the article of wear 10 is as permanently affixed, for example using mechanical (e.g. sewing) and/or chemical (e.g. permanent adhesive) means 19, such that the pad 12 is attached directly to the material 4 of the article of wear 10 in the desired location on the interior surface 15 (e.g. inside the crotch region of a panty). The permanent connection can be situated across the surface 18 of the pad 12 and/or about a periphery of the pad 12. In this embodiment, the pad 12 is considered part of the article of wear 10, and once the pad 12 becomes worn out after a limited number of washings, i.e. washed as the combination of the article of wear 10 and the pad 12, the article of wear 10 and attached pad 12 are disposed of together. In this manner, at least a portion of the pad 12 is affixed 19 to the article of wear 10 (e.g. such that the pad 12 can come into direct contact with the skin 16 of the wearer 8, for example).

In another embodiment, the pad 12 can be configured as releasably attached/coupled 19 with the article of wear 10. For example, this releasable coupling can be done via insertion (during wear of the article of wear 10 by the individual 8) of the pad 12 temporarily into an accessible enclosure 21 of the article of wear 10. Another option is to releasably attach the pad 12 to the article of wear via a releasable/reusable fastener 19 between the exterior surface 18 of the pad 12 and material 4 of the interior surface 15 of the article of wear 10. For example, the releasable fastener 19 can be Velcro™ (e.g. fabric hook-and-loop fasteners) for use in releasably securing the pad 12 to the article of wear 10, such that compatible strips of Velcro™ are secured to the surface 18 and the surface 15, thus facilitating releasable connection of the pad 12 in the interior of the article of wear 10. After use of the pad 12, the wearer 8 can remove the pad 12 from the article of wear 10 and wash/clean the pad 12 separately from the article of wear 10, as desired.

Also, the releasable fastener 19 can be referred to as a slidingly engaging fastener 19, where instead of loops and hooks, the releasable fastener 19 has interlocking islands with undercut edges that slide together (available at Material ConneXion). Other options for the releasable fastener 19 include means of fastening such as but not limited to snaps, buttons, zippers, etc.

Further to the above, in particular for a panty as the article of wear 10, the pad 12 can include the application of sticky adhesive 19 on the back surface 18 of the pad 12 to hold the pad 12 in place in the panty gusset 20. Another option is to include wings (e.g. material extensions, not shown) of the pad 12 which wrap around the material 4 of the panty 10, and for example snap together at the ends, providing additional stability.

### Absorbent Layer 2 Configuration

The absorbent non-woven layer 2 is formed by needle-punching and/or thermally bonding a combination of superabsorbent fibers 40, multi-component fibers 42 (e.g. polyethylene/polypropylene bi-component fibers) and rayon fibers 44 in predetermined amounts, as further described below. Methods for forming non-woven fabric layers 2 by needle-punching, such that the nonwoven layer 2 is created by mechanically orienting and interlocking the fibers 40,42,44 of a spunbonded or carded web. This mechanical interlocking of the fibers 40,42,44 is achieved with a plurality of barbed felting needles repeatedly passing into and out of the web (i.e. the mixture of fibers 40,42,44).

In terms of thermal-bonding, many materials can be used as a binder for thermally bonded nonwovens such as binding fibers, binding powder, and binding web. In terms of the binder polymer, properties include efficient melt flow, good adhesion to the carrier fiber, lower melting point than the carrier fiber, and desired stiffness or elasticity. Use of single-component and/or multi-component fibers, as binder fibers, can be used in thermal bonding of the nonwoven layer 2. With multi-component fibers 42, in thermal bonding, the higher melting portion of the fiber 42 maintains the integrity of the web, while the low melting point portion melts and will bond with other fibers at the fiber crossover points. Powdered polymers can be used in thermal bonding of the nonwoven layer 2, such as using powdered polyethylene. The powder can be applied between layers of fibers 40,42,44 when cross-laying, air laying, or as an after treatment. A short exposure in an oven is sufficient to melt and fuse the powder. For a binder web, an open-structured, low-melting-point thermoplastic fabric is placed between the webs and, during thermal bonding between calendar rolls, the fabric melts completely bonding the webs together. Thermoplastic coatings and hot melt print bonding can be used to control porosity in the layer 2, for example. Methods of thermal bonding include hot calendaring, belt calendaring, through-air thermal bonding, ultrasonic bonding, and/or radiant-heat bonding, etc.

In view of the above-described layers 2, it is recognised that the layer 2 can be comprised of a single (e.g. first) nonwoven layer 2. Alternatively, as further described below, the layer 2 can be comprised of a combination of multiple (e.g. a plurality) nonwoven sublayers SLn. The absorbent non-woven layer 2 may be a combination of multiple (e.g. two or three) sublayers SLn, wherein each sublayer Sln contains 50 to 100% by weight of the multi-component fibers 42, 0 to 50% by weight of the superabsorbent fibers 40, and 0 to 50% by weight of the rayon fibers 44, for a composite fiber total weight representing 100%. Accordingly, it is recognised that each of the sublayers SLn can contain, the same or different mixes/single fiber types, such as but not limited to: only multi-component fibers 42; a fiber mix of only multi-component fibers 42 and rayon fibers 44; a fiber mix of only multi-component fibers 42 and super absorbent fibers 40; a fiber mix of only rayon fibers 44 and super absorbent fibers 40; only rayon fibers 44; or only super absorbent fibers 40.

It is recognised that relative percentage composition of the different fiber types 40,42,44 in the sublayers SLn can be selected/defined so as to enhance absorption in the layer 2 by having fluid dispersion rates in the top/upper/inner layer(s) SLn (e.g. the respective sublayers SLn of the absorbent layer 2 closer to the wearer 8) greater than the fluid dispersion rates in the lower/outer layer(s) SLn (the respective sublayers SLn of the absorbent layer 2 further from the wearer 8) and thereby slowing down dispersion (and thus enhancing absorption/retention) though to the “outer” sublayer(s) of the absorbent layer 2. For example, the percentage composition of super absorbent fibers 40 can be higher in sublayers SLn further from the wearer 8 (e.g. closer to the layer 3) that in sublayers SLn closer to the wearer 8 (e.g. closer to the layer 1), in a three layer 1,2,3 composite pad 12.

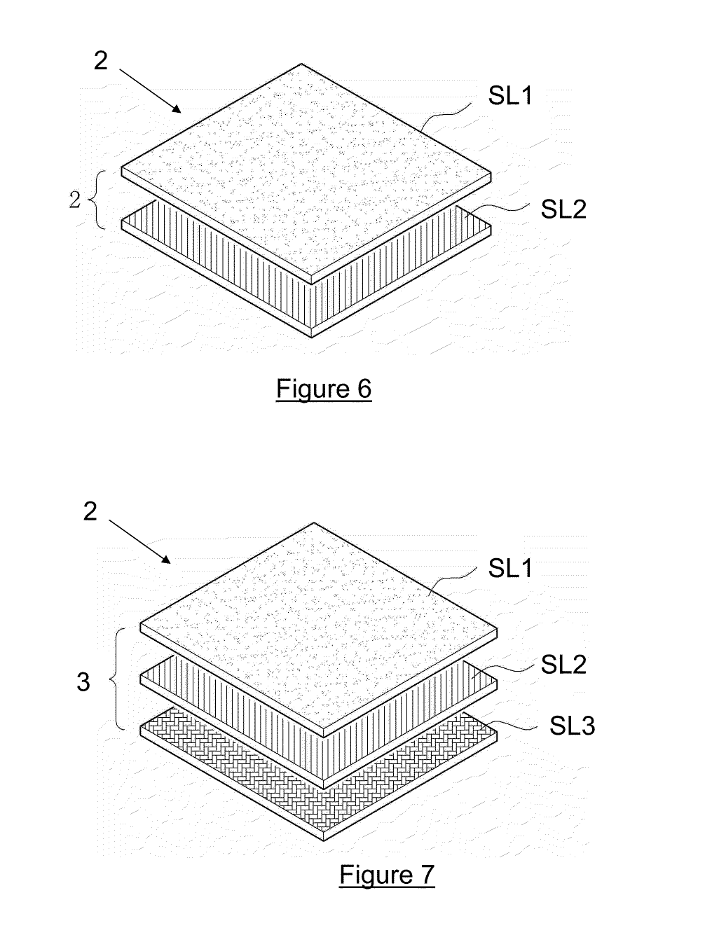

For example, in a two sublayer SLn layer 2, in FIG. 6, SL2 would have a higher percentage weight composition of respective super absorbent fibers 40 as compared to the percentage weight composition of respective super absorbent fibers 40 in SL1. For example, in a three sublayer SLn layer 2, in FIG. 7, SL2 would have a higher percentage weight composition of respective super absorbent fibers 40 as compared to the percentage weight composition of respective super absorbent fibers 40 in SL1, SL3 would have a higher percentage weight composition of respective super absorbent fibers 40 as compared to the percentage weight composition of respective super absorbent fibers 40 in SL2, and/or SL3 would have a higher percentage weight composition of respective super absorbent fibers 40 as compared to the percentage weight composition of respective super absorbent fibers 40 in SL1.

It is recognised that fiber types/configuration, other than the percentage amount of superabsorbent fibers 40 in the sublayer SLn, can contribute to the desired variable fluid dispersion rate properties between selected sublayers SLn. Accordingly, for example, in a two sublayer SLn layer 2, e.g. FIG. 6, SL2 would have a fiber configuration (e.g. percent composition of one or more selected fiber types and/or particular fiber porosity provided by selected fiber concentration/orientation/density via manufacturing of fiber placement in the sublayer) providing a higher fluid dispersion rate as compared to a fiber configuration providing a relatively (with respect to SL2) lower fluid dispersion rate in SL1. For example, in a three sublayer SLn layer 2, in FIG. 7, SL2 would have a fiber configuration providing a lower fluid dispersion rate as compared to a fiber configuration providing a relatively (with respect to SL2) higher fluid dispersion rate in SL1, SL3 would have a fiber configuration providing a lower fluid dispersion rate as compared to a fiber configuration providing a relatively (with respect to SL3) higher fluid dispersion rate in SL2, and/or SL3 would have a fiber configuration providing a lower fluid dispersion rate as compared to a fiber configuration providing a relatively (with respect to SL3) higher fluid dispersion rate in SL1. However, it is recognised that at least one of the sublayers SLn of the layer 2 contains superabsorbent fibers 40 (for example such that the total percentage weight of the superabsorbent fibers 40 in the entire layer 2 is greater than a selected total minimum percentage weight (e.g. 4%, 4.1%, 4.2%, 4.3%, 4.4.%, 4.5%, 4.6%, 4.7%, 4.8%, 4.9%, 5.0%, 5.1%, or 5.2%).

Accordingly, in view of the above, in the layer 2 (see FIGS. 5,6,7 by example only), the fluid dispersion rate of a first sublayer SLn that initially encounters the body fluid before a subsequent second sublayer SLn (which may or may not be adjacent to the first sublayer SLn in the stack of sublayers SLn of the layer 2) has a higher fluid dispersion rate than a second sublayer SLn that initially encounters the body fluid after the first sublayer SLn. Examples of this are such as but not limited to: the first sublayer is SL1 and the second sublayer is SL2; the first sublayer is SL1 and the second sublayer is SL3; the first sublayer is SL2 and the second sublayer is SL3; the first sublayer is SL1 and the second sublayer is SL4; the first sublayer is SL2 and the second sublayer is SL4; the first sublayer is SL3 and the second sublayer is SL4, etc.

Referring to FIGS. 4 and 5, shown is the absorbent layer 2 of the pad 12, having optional layers 1,3 (shown in ghosted view), such that the absorbent layer 2 is comprised of a plurality (e.g. more than one) sublayers SL1, SL2, SL3, SL4, . . . SLn. It is recognised that the sublayers SLn may be coupled to one another and/or may be mechanically separate from one another (i.e. unattached) at their surface interfaces Sln, other than for example at their peripheries 30 (see FIG. 3a,3b). For example, it is recognised that a majority portion of the fibers 40,42,44 of adjacent sublayers SLn may not be continuous (i.e. can be discontinuous) across the surface interface Sin between the sublayers SLn, such that each of the sublayers SLn have their own fibers in majority. The discontinuity in fiber strands between sublayers SLn can inhibit the phenomena of rewetting of a sublayer SLn closer to the wearer 8 by body fluid releases by the adjacent sublayer SLn further from the wearer 8.

It is also recognised that some of the fibers 40,42,44 of adjacent sublayers SLn may be somewhat continuous across the surface interface Sin due to surface roughness at the surface interface Sin caused by entanglement of fibers in one sublayer SLn with the adjacent fibers in the adjacent sublayer SLn, e.g. referred to as mechanical entanglement of fibers based on the role of frictional and/or attractive interfiber forces on fiber entanglement. This can also be referred to as interfiber friction (e.g. frictional interaction between fibers located in different sublayers SLn).

It is recognised that the porosity of each of the sublayers SLn can be different from one another, so as to increase the overall absorption rate of the body fluid into the absorbent layer 2, as compared to layers 2 that are single layered. The porosity of each of the layers SLn can be defined as a measure of the void spaces in the sublayer SLn material, and is a fraction of the volume of voids over the total volume of the fibers 40,42,44, e.g. between 0-1 or as a percentage between 0-100% porosity. For example, the porosity of a sublayer SLn is based on the percentage (for example measured by weight) of the super absorbent fibers 40 of the total amount of fibers 40,42,44 in the sublayer SLn, such that sublayers SLn with higher percentages of super absorbent fibers 40 are considered less porous than adjacent sublayers SLn with lower percentages of super absorbent fibers 40. For example, a sublayer SLn having 5% by weight of super fibers 40 has a higher relative porosity than a sublayer SLn having a 10% by weight of super fibers 40. For example, a subsequent sublayer SLn is less porous than a previous sublayer SLn closer to the wearer. For example, a subsequent sublayer SLn is more porous than a previous sublayer SLn closer to the wearer.

Referring again to FIGS. 4 and 5, in one embodiment for a multiple sublayer SLn containing absorbent layer 2 of the pad 12, at least one of the sublayers SLn contains multi-component fibers 42 as a majority by percentage weight of all the different fiber types (e.g. fibers 40, fibers 42, and/or fibers 44) in the sublayer SLn. It is recognised that the layer 2 is composed of multiple sublayers SLn, such that the relative percentage weights of the fibers 40, the fibers 42, and/or the fibers 44 for the total fiber weight in the respective sublayer SLn are the same or different for at least two of the sublayers SLn. In terms of reference for the sublayers SLn in respect to their stack position in the layer 2, the sublayer SLn closest to the wearer 8 (e.g. adjacent to the interior layer 1) is the first sublayer SLn and the sublayer SLn farthest from the wearer 8 (e.g. adjacent to the exterior layer 3) is the last sublayer SLn of the layer 2. Further, any sublayers SLn between the first and last sublayers SLn can be referred to as middle sublayers SLn.

Additional characteristics of the sublayers SLn of the absorbent layer 2 are features such as but not limited to:
(a) the sublayer SLn closer to the wearer 8 in the layer 2 than one of the other sublayers SLn (e.g. farther from the layer 3 and closer to the layer 1 of the layer 1,2,3, stack) has a lower percentage by weight of super absorbent fibers 40 of the total fiber weight in the respective sublayer SLn as compared to the other percentage by weight amounts of super absorbent fibers 40 of the total fiber weight(s) in at least one (or all) of the other sublayer(s) SLn of the layer 2;
(b) the percentage amount by weight of rayon fibers 44 is greater in the sublayer SLn closer to the wearer 8 than one of the other sublayers SLn (e.g. farther from the layer 3 and closer to the layer 1 of the layer 1,2,3, stack) relative to the other percentage by weight amounts of rayon fibers 44 of the total fiber weight(s) in at least one (or all) of the other sublayer(s) SLn of the layer 2;
(c) the percentage amount by weight of rayon fibers 44 is lower in the sublayer SLn closer to the wearer 8 than one of the other sublayers SLn (e.g. farther from the layer 3 and closer to the layer 1 of the layer 1,2,3, stack) relative to the other percentage by weight amounts of rayon fibers 44 of the total fiber weight(s) in at least one (or all) of the other sublayer(s) SLn of the layer 2;
(d) the sublayer SLn closer to the wearer 8 than one of the other sublayers SLn (e.g. closer to the layer 1 and farther from the layer 3 of the layer 1,2,3, stack) has a greater percentage by weight of multi-component fibers 42 of the total fiber weight in the respective sublayer SLn as compared to the other percentage by weight amounts of multi-component fibers 42 of the total fiber weight(s) in at least one (or all) of the other sublayer(s) SLn of the layer 2;
(e) the sublayer SLn further from the wearer 8 than one of the other sublayers SLn (e.g. closer to the layer 3 and farther from the layer 1 of the layer 1,2,3, stack) has the lower percentage by weight of multi-component fibers 42 of the total fiber weight in the respective sublayer SLn as compared to the other percentage by weight amounts of multi-component fibers 42 of the total fiber weight(s) in at least one (or all) of the other sublayer(s) SLn of the layer 2;
(f) the respective percentage weight of each sublayer SLn in the absorbent layer 2 of the total weight of the layer 2 is the same;
(g) the respective percentage weight of at least two sublayers SLn in the absorbent layer 2 of the total weight of the layer 2 are different, such that the respective percentage weight of the furthest sublayer SLn from the wearer 8 than one of the other sublayers SLn (e.g. closest to the layer 3 and farthest from the layer 1 of the layer 1,2,3, stack) is higher than each of the respective percentage weight(s) of the other sublayer(s) SLn in the layer 2.
(h) the percentage amount by weight of rayon fibers 44 is functionally absent (i.e. the minimal presence of the rayon fibers 44 in the sublayer SLn does not affect the absorbance and/or diffusion properties of the sublayer SLn as compared to other sublayers SLn that have a higher percentage by weight of rayon fibers 44) in the sublayer SLn closer to the wearer 8 than one of the other sublayers SLn (e.g. closest to the layer 1 and farthest from the layer 3 of the layer 1,2,3, stack) relative to the other percentage by weight amounts of rayon fibers 44 of the total fiber weight(s) in at least one (or all) of the other sublayer(s) SLn of the layer 2;
(i) the percentage by weight of the multi-component fibers 42 of the total fiber weight in the respective sublayer SLn is at least the same or greater than the percentage by weight of the super absorbent fibers 40 in the same respective sublayer SLn of the layer 2;
(j) the percentage by weight of the multi-component fibers 42 of the total fiber weight in the first sublayer SLn (e.g. closest to the layer 1 and farthest from the layer 3 of the layer 1,2,3, stack) is higher than the percentage by weight of the super absorbent fibers 40 in one or more of the other sublayer(s) SLn of the layer 2;
(k) the total percentage weight of the super absorbent fibers 40 combined from all of the sublayers SLn of the layer 2 is at least a selected percentage (e.g. 4%, 4.1%, 4.2%, 4.3%, 4.4%, 4.5%, 4.6%, 4.7%, 4.8%, 4.9%, 5.0%, 5.1%, 5.2%, or 5.3%) of the total fiber weight of the layer 2; and/or
(l) the layer 2 includes at least three sublayers SLn, such that the first and last sublayers SLn each contain less percentage amount by weight of rayon fibers 44 than one or more of the middle sublayers SLN between the first and last sublayers SLn.

A further embodiment of the cleanable and reusable body fluid-absorbing pad 12 is configured to move body fluid away from the skin of the wearer 8, such that the pad 12 includes the absorbent fibrous layer 2 comprising in total at least 5 wt % of synthetic superabsorbent fibers 40 configured to absorb the body fluid when present adjacent to the wearer 8 toward facing surface of the fibrous layer 2, the absorbent fibrous layer 2 formed by at least one of needle-punching, weaving, or thermally bonding of a fiber combination of at least two fiber types selected from the superabsorbent fibers 40, synthetic multi-component fibers 42, and rayon fibers 44.

For example, the layer 2 (or specified sublayer—e.g. SL1, SL2, SL3, SL4, SL5, SL6, etc.) can contain a majority (e.g. greater than 50%, 60%, 70%, 80%, 90%, or 95%) of the fibers (e.g. type 40 only, type 42 only, type 44 only, type 40 and 42 only, type 40 and 44 only, type 42 and 44, or type 40 and 42 and 44) combined as a non-woven mat (example fibrous quality). For example, the layer 2 (or specified sublayer—e.g. SL1, SL2, SL3, SL4, SL5, SL6, etc.) can contain a majority (e.g. greater than 50%, 60%, 70%, 80%, 90%, or 95%) of the fibers (e.g. type 40 only, type 42 only, type 44 only, type 40 and 42 only, type 40 and 44 only, type 42 and 44, or type 40 and 42 and 44) combined as a woven mat (example fibrous quality). It is also recognised that the layer 2 (or specified sublayer—e.g. SL1, SL2, SL3, SL4, SL5, SL6, etc.) can be 100% woven (example fibrous quality). It is also recognised that the layer 2 (or specified sublayer—e.g. SL1, SL2, SL3, SL4, SL5, SL6, etc.) can be 100% non-woven (example fibrous quality).

Further, the fibrous quality (i.e. woven, non-woven, or a combination of woven and non-woven) of one or more selected sublayer(s) SLn in the layer 2 can be made as described above for the layer 2. Further, it is recognised that the fibrous quality for a sublayer SLN can be the same or different from the fibrous quality of other(s) of other sublayer(s) SLn in the layer 2.

### Example Configurations of the Sublayers SLn

As described above, the layer 2 absorbs body fluids transported from the diffusive fabric layer 1 (if present in the pad 12) and tries to retain the body fluids once absorbed. The absorbent non-woven layer 2 is formed by needle-punching or thermally bonding superabsorbent fibers 40, multi-component fibers 42, and/or rayon fibers 44, and can comprise one or more sublayers SLn (see FIG. 5). For example, in FIG. 1 is shown the form of mono-layer 2, in FIG. 6 is shown a di-sublayer SLn and in FIG. 7 is shown a tri-sublayer SLn layer 2. Applicant has determined that the absorbent non-woven layer 2 including multiple (e.g. two or three) sublayers SLn can improve rewetting properties of the layer 2, as further described below.

Where the absorbent non-woven layer 2 comprises sublayers SLn in the form of a combination of two or three layers, the respective sublayers SL1, SL2, and SL3 can comprise a polyethylene/polypropylene bi-component fibers 42 wherein the bi-component fiber 42 is present in an amount of 50 to 100 wt %, based on the total weight of the corresponding sublayer SLn, comprises 0 to 50% by weight of a superabsorbent fibers 40 of the corresponding sublayer SLn, and 0 to 50% by weight of a rayon fibers 44 of the corresponding sublayer SLn, based on the total weight of the corresponding sublayer SLn. The super-absorbent fibers 40 are present in an amount of 5% by weight or higher, for example, based on the total weight of the sublayers SLn (e.g. the layer 2).

### Example Layer 2 Configurations

### EXAMPLE 1

An Aquatrans® fabric (0.7 mm, 150 g/m2) available from Welcron Co., Ltd. was used as a diffusive fabric layer 1. A non-woven fabric 2 obtained by needle-punching a combination of 5% by weight of a superabsorbent fiber 40 (Oasis® available from TAL Co., Ltd., also, in the following Examples), 50% by weight of polyethylene/polypropylene bi-component fiber 42 S/S 3D (polypropylene 50 wt %, polyethylene 50 wt %, and EZBON-H available from Woongjin Chemical Co., Ltd., also, in the following Examples), and 45% by weight of rayon fibers 44 (available from Lenzing fibers Inc. of Austria, also, in the following Examples) was used in the form of non-woven monolayer 2 (thickness: 1.5 mm, weight: 180 g/m2) as an absorbent non-woven layer 2. A permeable film 3 available from Daemyung chemical Co., Ltd, (thickness: 0.3 mm, weight: 20 g/m2, also, in the following Examples) was used as a waterproof film layer 3. These three layers were stacked and sewed on their periphery 30 (see FIG. 3b) to prepare an absorbent fabric pad 12. The total thickness of this pad 12 product was 2.8 mm.

### EXAMPLE 2

An absorbent fabric pad 12 was prepared in the same manner as in Example 1 except that an absorbent non-woven layer 2 comprised two sublayers SL1, SL2 as follows. The first sublayer SL1 was prepared from a combination of polyethylene/polypropylene bi-component fiber 42 of S/S 3D 50 wt %, superabsorbent fibesr 40 of 25 wt % and rayon fibers 44 of 5 wt %. The second sublayer SL2 was prepared from a combination of polyethylene/polypropylene bi-component fibers 42 of S/S 3D 50 wt % and superabsorbent fibers 40 of 50 wt %. The first and second sublayers SL1,SL2 were present in an amount of 35% and 65%, based on the total weight of the absorbent non-woven layer 2, respectively.

### EXAMPLE 3

An absorbent fabric pad 12 was prepared in the same manner as in Example 1 except that an absorbent non-woven layer 2 comprised two sublayers SL1, SL2 as follows. The first sublayer Sl1 was prepared from a combination of polyethylene/polypropylene bi-component fiber 42 of S/S 6D (polypropylene 50 wt %, polyethylene 50 wt %, EZBON-H available from Woongjin Chemical Co., Ltd., also, in the following Examples) 50 wt %, superabsorbent fibers 40 of 10 wt %, and rayon fibers 44 of 40 wt %. The second sublayer Sl2 was prepared from a combination of polyethylene/polypropylene bi-component fiber 42 of S/S 3D 50 wt %, superabsorbent fibers 40 of 20 wt % and rayon fibers 44 of 30 wt %. The first and second sublayers SL1, SL2 were present in an amount of 50% and 50%, based on the total weight of the absorbent non-woven layer 2, respectively.

### EXAMPLE 4

An absorbent fabric pad 12 was prepared in the same manner as in Example 1 except that an absorbent non-woven layer 2 comprised two sublayers SL1,SL2 as follows. A first sublayer SL1 was prepared from only polyethylene/polypropylene bi-component fibers 42 S/S 3D (100% wt). A second sublayer Sl2 was prepared from a combination of polyethylene/polypropylene bi-component fibers 42 of S/S 3D 50 wt %, and superabsorbent fibers 40 of 50 wt %. The first and second sublayers SL1,SL2 were present in differing amounts of 35% and 65%, based on the total weight of the absorbent non-woven layer 2, respectively.

### EXAMPLE 5

An absorbent fabric pad 12 was prepared in the same manner as in Example 1 except that an absorbent non-woven layer 2 comprised three sublayers SL1,SL2,SL3, as follows. A first sublayer SL1 was prepared from only polyethylene/polypropylene bi-component fibers 42 of S/S 6D (100% wt). A second sublayer SL2 was prepared from a combination of polyethylene/polypropylene bi-component fibers 42 of S/S 3D 50 wt % and rayon fibers 44 of 50 wt %. A third sublayer SL3 was prepared from a combination of polyethylene/polypropylene bi-component fibers 42 of S/S 3D 50 wt % and superabsorbent fibers 40 of 50 wt %. The first, second and third sublayers SL1,SL2,SL3 were present in differing amounts of 35%, 30% and 40%, based on the total weight of the absorbent non-woven layer 2, respectively.

### EXAMPLE 6

An absorbent fabric pad 12 was prepared in the same manner as in Example 1 except that an absorbent non-woven layer 2 comprised three sublayers SL1,SL2,SL3, as follows. A first sublayer Sl1 was prepared from only polyethylene/polypropylene bi-component fibers 42 of S/S 6D 100 wt %. A second sublayer SL2 was prepared from a combination of polyethylene/polypropylene bi-component fibers 42 of S/S 3D 80 wt % and rayon fibers 44 of 20 wt %. A third sublayer SL3 was prepared from a combination of polyethylene/polypropylene bi-component fibers 42 of S/S 3D 50 wt % and superabsorbent fibers 40 of 50 wt %. The first, second and third sublayers SL1,SL2,SL3 were present in differing amounts of 35%, 30% and 40%, based on the total weight of the absorbent non-woven layer 2, respectively.

In view of the above example pad 12 configurations, the absorption rate and rewetting properties of the absorbent pads 12 thus prepared were tested. The test for the absorption rate of the different layers 2 was carried out by dropping 5 ml of artificial urine (0.9% NaCl) in each pad and measuring the time (sec/5 ml) taken for the pad 12 to absorb the artificial urine (e.g. absorption rate increases as absorption time becomes shorter). The test for rewetting properties was carried out by measuring the amount (g) of body fluid leaked from a tissue, when a load of 6 kg is applied to the pad 12 for one minute after absorption.

TABLE 1

| | Absorption rate (sec/5 ml) | Rewet (g) |
|---|---|---|
| Ex. 1 | 6.52 | 2.80 |
| Ex. 2 | 6.24 | 1.95 |
| Ex. 3 | 5.30 | 1.98 |
| Ex. 4 | 5.21 | 2.06 |
| Ex. 5 | 4.84 | 1.40 |
| Ex. 6 | 4.60 | 1.56 |

In view of the above tabular results, it can be seen that the absorbent fabric pad 12 using the absorbent non-woven layer comprising three sublayers SLn (Example 6) exhibited the most superior absorption rate. Rewetting, the phenomenon wherein absorbed moisture is leaked out was excellent in all absorbent non-woven layers having a multilayer 2 structure (Examples 2 to 6), as compared to the single layer 2 structure. As such, the absorbent non-woven layer 2 was composed of several sublayers SLn made of different fiber types combinations, thus facilitating relative decreases in the rewetting phenomenon.

### Further Example Absorbent Layer 2 Configurations

Described below are other multiple layer SLn configurations. It is recognised that the provided percent weight values and ranges of the table can be in the specific layers SLn provided and/or can used to define other layers SLn of a multi-layer SLn absorbent layer 2 having fewer or more than the three layers SL3 provided below in the table, by way of example only.

For example, the example composition and/or composition ranges of the sub layer SL1 can be used to define the composition and/or composition ranges of another sublayer SLn in the layer 2, for example in sub layer SL2, present in a selected multi-layer configuration of the layer 2 (some or which are not shown), for example in sub layer SL2, for example in sub layer SL3, for example in sub layer SL4, for example in sub layer SL5, for example in sub layer SL6, for example in sub layer SL7, for example in sub layer SL8, for example in sub layer SL9, for example in sub layer SL10.

For example, the example composition and/or composition ranges of the sub layer SL2 can be used to define the composition and/or composition ranges of another sublayer SLn in the layer 2, for example other sub layer(s) SL1, 3, 4, 5, 6, 7, 8, 9, and/or 10 present in a selected configuration of the layer 2 (some or which are not shown), for example in sub layer SL1, for example in sub layer SL3, for example in sub layer SL4, for example in sub layer SL5, for example in sub layer SL6, for example in sub layer SL7, for example in sub layer SL8, for example in sub layer SL9, for example in sub layer SL10.

For example, the example composition and/or composition ranges of the sub layer SL3 can be used to define the composition and/or composition ranges of another sublayer SLn in the layer 2, for example other sub layer(s) SL1, 2, 4, 5, 6, 7, 8, 9, and/or 10 present in a selected configuration of the layer 2 (some or which are not shown), for example in sub layer SL1, for example in sub layer SL2, for example in sub layer SL4, for example in sub layer SL5, for example in sub layer SL6, for example in sub layer SL7, for example in sub layer SL8, for example in sub layer SL9, for example in sub layer SL10.

Table 1 of example sub layer SLn configurations by percent weight composition of the components 40, 42, 44 with respect to one another for the complete weight total of the layer 2 (i.e. all percentages of each of the components 40,42,44 in each of the layers adds up to 100% for the total weight of the layer 2). Also shown is the sub total percent weight of each of the sub layers SLn as a sub total percentage for the layer of the total weight of the layer 2. as noted, “SAF” refers to super absorbent fibers 40, “PE” and “PP” refer to different embodiments of the multi-component fibers 42, and “RAYON” refers to the rayon 44.

TABLE 1

| LAYER | | | | | | | | | | | |
|---|---|---|---|---|---|---|---|---|---|---|---|
| No. | MATERIAL | 1 | 2 | 3 | 4 | 5 | 6 | 7 | 8 | 9 | 10 |
| 1^{st} LAYER | SAF | 2% | 4% | 10% | 8% | 5% | 7% | 6% | 8% | 10% | 8% |
| | PE | 15% | 12% | 5% | 13% | 10% | 8% | 4% | 6% | 5% | 9% |
| | PP | 13% | 10% | 15% | 18% | 15% | 10% | 5% | 9% | 8% | 11% |
| | RAYON | 10% | 8% | 8% | 3% | 9% | 5% | 5% | 8% | 6% | 5% |
| | SUB TOTAL | 40% | 34% | 38% | 42% | 39% | 30% | 20% | 31% | 29% | 33% |
| 2^{nd} LAYER | SAF | 15% | 14% | 8% | 9% | 5% | 8% | 5% | 4% | 5% | 8% |
| | PE | 5% | 8% | 10% | 8% | 5% | 6% | 6% | 8% | 11% | 6% |
| | PP | 2% | 10% | 5% | 4% | 7% | 10% | 9% | 9% | 5% | 9% |
| | RAYON | 10% | 5% | 10% | 10% | 12% | 5% | 15% | 13% | 15% | 5% |
| | SUB TOTAL | 32% | 37% | 33% | 31% | 29% | 29% | 35% | 34% | 36% | 28% |
| 3rdLAYER | SAF | 3% | 2% | 2% | 3% | 10% | 5% | 9% | 8% | 5% | 4% |
| | PE | 5% | 5% | 10% | 4% | 10% | 11% | 15% | 11% | 9% | 10% |
| | PP | 10% | 5% | 5% | 3% | 3% | 5% | 11% | 7% | 12% | 5% |
| | RAYON | 10% | 17% | 12% | 17% | 9% | 20% | 10% | 9% | 9% | 20% |
| | SUB TOTAL | 28% | 29% | 29% | 27% | 32% | 41% | 45% | 35% | 35% | 39% |
| TOTAL | SAF | 20% | 20% | 20% | 20% | 20% | 20% | 20% | 20% | 20% | 20% |
| | PE | 25% | 25% | 25% | 25% | 25% | 25% | 25% | 25% | 25% | 25% |
| | PP | 25% | 25% | 25% | 25% | 25% | 25% | 25% | 25% | 25% | 25% |
| | RAYON | 30% | 30% | 30% | 30% | 30% | 30% | 30% | 30% | 30% | 30% |

Further to the above, it is recognised that for one sub layer SLn (e.g. the first sub layer SL1), the component 40 can have the following percent weight (of the total layer 2) ranges 2-5,2-4,6-10,6-8, and/or 8-10, the component 42 can have the following percent weight (of the total layer 2) ranges 9-20, 9-11, 11-13, 13-15, 15-17, 17-19, 19-20, 20-31, 20-22, 22-24,24-26,26-28,28-30, and/or 30-31, the component 44 can have the following percent weight (of the total layer 2) ranges 3-10,3-5,5-7,7-9,and/or 9-10, and the percent weight subtotal for the sublayer SLn is in the range of 20-42,20-30,30-42,20-22,22-24,24-26,26-28,28-30,30-32,32-34,34-36,36-38,38-40,and/or 40-42.

Further to the above, it is recognised that for one sub layer SLn (e.g. the second sub layer SL2), the component 40 can have the following percent weight (of the total layer 2) ranges 5-15,5-9,5-7,7-9,9-11,11-13,and/or 13-15, the component 42 can have the following percent weight (of the total layer 2) ranges 7-18, 7-9, 9-11, 12-18, 12-14, 14-16, and/or 16-18, the component 44 can have the following percent weight (of the total layer 2) ranges 5-15,5-10,7-10,10-15,10-11,11-12,12-13,13-14,and/or 14-15, and the percent weight subtotal for the sublayer SLn is in the range of 28-37, 28-30,30-32,32-34,34-36,and/or 36-37.

Further to the above, it is recognised that for one sub layer SLn (e.g. the second sub layer SL3), the component 40 can have the following percent weight (of the total layer 2) ranges 2-10,2-5,5-8,8-10,5-6,6-7,7-8,8-9,and/or 9-10, the component 42 can have the following percent weight (of the total layer 2) ranges 7-10,10-18,18-26,13-18,10-12,12-14,14-16,and/or 16-18, the component 44 can have the following percent weight (of the total layer 2) ranges 9-12,12-17,17-20,10-13,13-15,15-17,17-19, and the percent weight subtotal for the sublayer SLn is in the range of 27-45, 27-31,31-35,35-40,40-45,29-31,31-33,33-35,and/or 35-37.

In any event, it is recognised that the actual values of each of the components 40,42,44 in the sub layer SLn can be selected from the above provided example ranges/values to combine to represent one of the values in the range for the percent weight subtotal for the sublayer SLn (e.g. a selection of 3 from the range 2-5 with a selection of 22 from the range of 20-31 with a selection of 6 from the range of 5-7 would result in a percent weight subtotal—i.e. 31—in the subtotal range of 30-32). Accordingly, the layer 2 can contain at least one of the above described sub layers SLn with respective selected component 40,42,44 ranges and the corresponding matching subtotal range form the ranges provided by example.

Further, it is recognised that the percent weight subtotal ranges for each of the sublayers SLn can be selected as any combination of the above-provided ranges, such that the total weight would add to 100 percent for a potential number from each of the ranges (for example, subtotal ranges of 30-35,31-37, and 24-33 provide for the actual potential respective numbers to be 34,35,31 from the rangers for the 100 percent total).

It is also recognised that the provided numerical approximate values. For example, the number 10 can be used to represent the nearest adjacent values between 9.9 and 10.1. Therefore, a range specified as 10-12 can actually encompass the range of 9.9 to 12, 10-12.1, 9.9 to 12.1 unless the selected number limit of any of the above-provided ranges is defined as an absolute limit (e.g. the range 10-12 where 10 is absolute means 10-12/12.1 such that range limit 10 is for only 10 and above for the specified range, or the range 10-12 where 12 is absolute means 9.9/10-12 such that range limit 12 is for only 12 and below in the specified range.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A cleanable and reusable body fluid-absorbing pad to move body fluid away from the skin of a wearer, the pad including:
an absorbent non-woven layer comprising in total at least 5 wt % of synthetic superabsorbent fibers configured to absorb the body fluid when present adjacent to a wearer toward facing surface of the non-woven layer, the absorbent non-woven layer formed by at least one of needle-punching or thermally bonding of a fiber combination of at least two fiber types selected from the superabsorbent fibers, synthetic multi-component fibers, and rayon fibers.

2. The pad of claim 1 further comprising a water resistant film adjacent to a wearer away facing surface of the absorbent non-woven layer and exhibiting resistance to the transfer of the body fluid through the film, wherein the absorbent non-woven layer and the film are stacked and coupled to form the pad.

3. The pad of claim 2 further comprising a diffusive fabric layer configured for diffusing the body fluid through the diffusive fabric layer and away from contact with the skin of the wearer, the diffusive fabric layer situated adjacent to the wearer toward facing surface of the absorbent non-woven layer, wherein the absorbent non-woven layer and the diffusive fabric layer and the film are stacked and coupled to form the pad.

4. The pad of claim 3, wherein the diffusive fabric layer is formed by a weight loss finishing process as a double weave structure having one surface woven with a polyethyleneterephthalate (PET) filament and the other surface woven with a divided PET/nylon-conjugated fiber.

5. The pad of claim 2, wherein the synthetic multi-component fibers are bi-component fibers.

6. The pad of claim 5, wherein the bi-component fibers are composed of polyethylene and polypropylene.

7. The pad of claim 2, wherein the absorbent non-woven layer is comprised of a plurality of non-woven sublayers positioned adjacent to one another.

8. The pad of claim 7, wherein the interface surface of adjacent non-woven sublayers are configured to inhibit rewetting of a first sublayer closer to the wearer with body fluid released from a second adjacent sublayer further from the wearer.

9. The pad of claim 8, wherein the plurality of non-woven sublayers are mechanically separate from one another at their respective surface interfaces, such that a majority portion of fiber strands in the adjacent sublayers are discontinuous with respect to one another across their corresponding surface interface.

10. The pad of claim 9, wherein all of the fiber strands are discontinuous with respect to one another across their corresponding surface interface.

11. The pad of claim 2 affixed to an article of wear.

12. The pad of claim 11, wherein the article of wear is an undergarment and the body fluid is urine.

13. The pad of claim 2 configured for releasable securing to an article of wear by at least one of a mechanical fastener, a chemical fastener, or insertion into an enclosure of the article of wear.

14. The pad of claim 13, wherein the article of wear is protective equipment and the body fluid is perspiration.

15. The pad of claim 3 further comprising the diffusive fabric layer and the non-woven layer affixed to one another at their surface interface at a plurality of coupling locations distributed over the surface interface.

16. The pad of claim 15, wherein the plurality of distributed coupling locations is in the form of a grid that includes at least one of regular or random spacing of the coupling locations.

17. The pad of claim 16, wherein the spacing is a predefined minimum distance to promote fluid transfer of the body fluid between the diffusive fabric layer and the non-woven layer by minimizing a gap distance there-between.

18. The pad of claim 17, wherein an adhesive is deposited in at least some of the distributed coupling locations.

19. The pad of claim 17, wherein a stitch between the layers is formed in at least some of the distributed coupling locations.

20. The pad of claim 19, wherein the stitches are inserted through the diffusive fabric layer and only partway through the thickness of the non-woven layer.

21. The pad of claim 7, wherein at least two of the sublayers contains different amounts of the fiber types.

22. The pad of claim 21, wherein the different amount of the fiber types is selected from the group comprising: only multi-component fibers; a fiber mix of only multi-component fibers and rayon fibers; a fiber mix of only multi-component fibers and super absorbent fibers ; a fiber mix of only rayon fibers and super absorbent fibers; only rayon fibers; and only super absorbent fibers.

23. The pad of claim 22, wherein the relative percentage weights of the fiber types are different for at least two of the sublayers of the plurality of sublayers.

24. The pad of claim 23, wherein a selected sublayer closer to the wearer has a lower percentage by weight of super absorbent fibers 40 of the total fiber weight in a selected sublayer as compared to the other percentage by weight of super absorbent fibers of the total fiber weight in at least one of the other sublayers farther from the wearer than the selected sublayer.

25. The pad of claim 23, wherein the percentage by weight of rayon fibers is greater in a selected sublayer as compared to the other percentage by weight of rayon fibers of the total fiber weight in at least one of the other sublayers farther from the wearer than the selected sublayer.

26. The pad of claim 23, wherein the percentage amount by weight of rayon fibers is lower in a selected sublayer as compared to the other percentage by weight of rayon fibers of the total fiber weight in at least one of the other sublayers farther from the wearer than the selected sublayer.

27. The pad of claim 23, wherein a selected sublayer has a greater percentage by weight of multi-component fibers of the total fiber weight in the selected sublayer as compared to the other percentage by weight of multi-component fibers of the total fiber weight in at least one of the other sublayers farther from the wearer than the selected sublayer.

28. The pad of claim 23, wherein a selected sublayer has a lower percentage by weight of multi-component fibers of the total fiber weight in the respective selected sublayer as compared to the other percentage by weight of multi-component fibers of the total fiber weight in at least one of the other sublayers farther from the wearer than the selected sublayer.

29. The pad of claim 23, wherein the respective percentage weight of each sublayer in the absorbent non-woven layer of the total weight of the absorbent non-woven layer is the same.

30. The pad of claim 23, wherein the respective percentage weight of at least two selected sublayers in the absorbent non-woven layer of the total weight of the absorbent non-woven layer are different, such that the respective percentage weight of the furthest sublayer from the wearer is higher than each of the respective percentage weight of the other sublayers in the absorbent non-woven layer.

31. The pad of claim 23, wherein the percentage amount by weight of rayon fibers is functionally absent in a selected sublayer closer to the wearer relative to the other percentage by weight of rayon fibers of the total fiber weight in at least one of the other sublayers farther from the wearer than the selected sublayer.

32. The pad of claim 23, wherein the percentage by weight of the multi-component fibers of the total fiber weight in a selected sublayer is at least the same or greater than the percentage by weight of the super absorbent fibers in the same selected sublayer.

33. The pad of claim 23, wherein the percentage by weight of the multi-component fibers of the total fiber weight in a first sublayer is higher than the percentage by weight of the super absorbent fibers in one or more of the other sublayers.

34. The pad of claim 23, wherein the total percentage weight of the super absorbent fibers 40 combined from all of the sublayers is greater than a percentage selected from the group comprising; 4%, 4.1%, 4.2%, 4.3%, 4.4%, 4.5%, 4.6%, 4.7%, 4.8%, 4.9%, 5.0%, 5.1%, 5.2%, or 5.3% of the total fiber weight of the absorbent non-woven layer.

35. The pad of claim 23, wherein the absorbent non-woven layer includes at least three sublayers, such that the first and last sublayers each contain less percentage by weight of rayon fibers than one or more middle sublayers between the first and last sublayers.

36. The pad of claim 21, wherein a porosity of each of the sublayers is different from one another.

37. The pad of claim 36 the porosity of a selected sublayer is based on a percentage by weight of the super absorbent fibers of a total weight of fibers in the selected sublayer, such that a sublayer of the plurality of sublayers with a higher percentage of super absorbent fibers is less porous than another sublayer of the plurality of sublayers with a lower percentage of super absorbent fibers, respectively.

38. The pad of claim 37, wherein the another sublayer is less porous than the selected sublayer situated closer to the wearer than the another sublayer.

39. The pad of claim 37, wherein the another sublayer is more porous than the selected sublayer situated closer to the wearer than the another sublayer.

40. The pad of claim 8, wherein a first fluid dispersion rate of the first sublayer is greater than a second fluid dispersion rate of the second sublayer.

41. The pad of claim 39, wherein the super absorbent fibers include polymer of fibrous form that are insoluble in the body fluid but swellable when they absorb the body fluid.

42. The pad of claim 41, wherein the super absorbent fibers contribute to the cohesiveness of the fibers with respect to one another in the non-woven layer.

43. The pad of claim 42, wherein the non-woven layer also contains woven fibers.

44. The pad of claim 43, wherein the woven fibers include a fiber type selected from the group comprising: the superabsorbent fibers; the synthetic multi-component fibers; and the rayon fibers.

45. A body fluid-absorbing fabric pad to move moisture including sweat and urine away from the skin of a wearer, the body fluid-absorbing fabric pad comprising:
a diffusive fabric layer coming into contact with the skin of the wearer to remove moisture from the skin;
an absorbent non-woven layer coming into contact with the diffusive fabric layer, the absorbent non-woven layer comprising at least 5 wt % of a superabsorbent fiber to remove moisture transported from the diffusive fabric layer; and
a waterproof film coming into contact with the absorbent non-woven layer and exhibiting waterproofing properties and breathability,
wherein the diffusive fabric layer is formed by weight loss finishing process a double weave structure having one surface woven with a polyethyleneterephthalate (PET) filament and the other surface woven with a divided PET/nylon-conjugated fiber,
wherein the absorbent non-woven layer is formed by needle-punching or thermally bonding a superabsorbent fiber, a polyethylene/polypropylene bicomponent fiber and a rayon fiber, and
wherein the diffusive fabric layer, the absorbent non-woven layer and the waterproof film are stacked and sewed to form the body fluid-absorbing fabric pad.

46. The body fluid-absorbing fabric pad according to claim 45, wherein the absorbent non-woven layer is a monolayer containing 5 to 50% by weight of the superabsorbent fiber, 50 to 70% by weight of the polyethylene/polypropylene bicomponent fiber and 20 to 45% by weight of the rayon fiber.

47. The body fluid-absorbing fabric pad according to claim 45, wherein the absorbent non-woven layer is a combination of two or three sublayers,
wherein each sublayer contains 50 to 100% by weight of the polyethylene/polypropylene bicomponent fiber, 0 to 50% by weight of the superabsorbent fiber, and 0 to 50% by weight of the rayon fiber.

48. A cleanable and reusable body fluid-absorbing pad to move body fluid away from the skin of a wearer, the pad including:
an absorbent fibrous layer comprising in total at least 5 wt % of synthetic superabsorbent fibers configured to absorb the body fluid when present adjacent to a wearer toward facing surface of the fibrous layer, the absorbent fibrous layer formed by at least one of needle-punching, weaving, or thermally bonding of a fiber combination of at least two fiber types selected from the superabsorbent fibers, synthetic multi-component fibers, and rayon fibers.

49. The pad of claim 48, wherein the absorbent fibrous layer is comprised of a plurality of sublayers positioned adjacent to one another.

50. A pad of claim 48, wherein the absorbent fibrous pad contains a majority of the fibers combined as a non-woven mat for defining a fibrous quality of the layer 2.
